(19) United States
(12) Patent Application Publication
(10) Pub. No.: US 2025/0387072 A1
(43) Pub. Date: Dec. 25, 2025

(54) STROKE DETECTION SYSTEM

(71) Applicant: Covidien LP, Mansfield, MA (US)

(72) Inventors: Arun Kumar Sathiyamoorthy, Chennai (IN); Randal C. Schulhauser, Phoenix, AZ (US)

(21) Appl. No.: 19/243,159

(22) Filed: Jun. 19, 2025

Related U.S. Application Data
(60) Provisional application No. 63/662,292, filed on Jun. 20, 2024.

(51) Int. Cl. A61B 5/00; A61B 5/026; A61B 5/291; A61B 5/31; A61B 5/369

(52) U.S. Cl. CPC A61B 5/4076; A61B 5/0095; A61B 5/026; A61B 5/291; A61B 5/31; A61B 5/369; A61B 5/6862; A61B 5/746

(57) ABSTRACT

A medical system configured to detect and/or monitor one or more physiological parameters of the patient including an electroencephalogram (EEG) of the patient, a oxygen level of the blood patient (e.g., a jugular venous oxygen saturation (SjVO2)), and/or a blood flow of the patient (e.g., a cerebral blood flow). The medical system may include processing circuitry configured to monitor the physiological parameters and compare the physiological parameters to one or more thresholds. The medical system may be configured to indicate that the patient may have experienced a stroke based on the comparison of the physiological parameters with the thresholds. In examples, the processing circuitry is configured to indicate a type of stroke the patient may have experienced based on the comparison.

### CROSS-REFERENCE TO RELATED APPLICATIONS

This application claims the benefit of and priority to U.S. Provisional Patent Application Ser. No. 63/662,292 filed Jun. 20, 2024, the entire disclosure of which is incorporated by reference herein.

### TECHNICAL FIELD

The present disclosure generally relates to strokes, and to stroke detection.

### BACKGROUND

Stroke is a medical condition that can cause permanent neurological damage, complications, and death. Stroke may be characterized as the rapidly developing loss of brain functions due to a disturbance in the blood vessels supplying blood to the brain. The loss of brain functions can be a result of ischemia (lack of blood supply) caused by thrombosis or embolism. During a stroke, the blood supply to an area of a brain may be decreased, which can lead to dysfunction of the brain tissue in that area.

Detecting and treating strokes as soon as possible promotes the effectiveness of stroke therapy received by patients. A variety of approaches exist for treating patients undergoing a stroke. For example, a clinician may administer anticoagulants or may undertake intravascular interventions such as thrombectomy procedures to treat ischemic stroke. However, such treatments may be frequently underutilized and/or relatively ineffective due to the failure to timely identify whether a patient is undergoing or has recently undergone a stroke.

### SUMMARY

A medical system configured to detect and/or monitor physiological parameters of a patient using a stent. The medical system may be configured to monitor one or more physiological parameters of the patient including an electroencephalogram (EEG) of the patient, an oxygen level of the blood patient (e.g., a jugular venous oxygen saturation (SjVO2)), and/or a blood flow of the patient (e.g., a cerebral blood flow). Processing circuitry may be configured to monitor the physiological parameters and compare the physiological parameters to one or more thresholds. The processing circuitry may be configured to indicate that the patient may have experienced a stroke based on the comparison of the physiological parameters with the thresholds. In examples, the processing circuitry is configured to indicate a type of stroke the patient may have experienced based on the comparison.

In an example, a medical system includes: a stent configured to expand within a blood vessel of a patient, the stent comprising: a skeleton configured to define a lumen when the skeleton is positioned within the blood vessel, wherein the stent is configured to allow the blood in the vessel to flow in a direction along a longitudinal axis extending from a proximal portion of the stent to a distal portion of the stent when the skeleton positions within the blood vessel; one or more conductive elongate fibers supported by the skeleton and extending from the proximal portion to the distal portion, wherein the one or more conductive elongate fibers are configured to receive an electrical signal indicative of a electroencephalogram of the patient when the skeleton positions within the blood vessel; and processing circuitry configured to receive a signal indicative of the electrical signal from the one or more conductive elongate fibers, wherein the processing circuitry is configured to determine the electroencephalogram using the signal.

In an example, a medical system includes: a stent configured to expand within a blood vessel a patient, the stent comprising: a skeleton configured to define a lumen when the skeleton is positioned within the blood vessel; a conductive material supported by the skeleton, wherein the conductive material is configured to receive and conduct an electrical signal indicative of a electroencephalogram (EEG) of the patient when the skeleton positions within the blood vessel; a light emitter device supported by the skeleton and positioned within the lumen, wherein the light emitter device is configured to cause an photoacoustic response of some portion of the blood in the vessel when the light emitter device projects light into the portion of the blood, and wherein the photoacoustic response is indicative of an oxygen level of the portion of the blood; and a first material supported by the skeleton, wherein the first material is configured to develop a first electric potential in response to a first mechanical stress generated by impingement of a pressure wave on the first material caused by the photoacoustic response, wherein the stent is configured to communicate a EEG signal indicative of the electrical signal from the conductive material to processing circuitry, communicate a first signal indicative of the first electric potential from the first material to the processing circuitry, and communicate a flow signal indicative of a blood flow rate in the blood vessel to the processing circuitry, and wherein the stent is configured to allow the blood in the vessel to flow in a direction along a longitudinal axis extending from a proximal portion of the stent to a distal portion of the stent when the skeleton positions within the blood vessel.

In an examples, a method comprises: supporting, using a skeleton of a stent configured to define a lumen when the skeleton is positioned within a blood vessel of a patient, one or more conductive elongate fibers extending from a proximal portion of the stent to a distal portion of the stent, wherein the stent is configured to allow the blood in the blood vessel to flow in a direction along a longitudinal axis extending from the proximal portion to the distal portion when the skeleton is positioned in the blood vessel; receiving, using the one or more conductive fibers, an electrical signal indicative of a electroencephalogram (EEG) of the patient when the skeleton positions in the blood vessel; and providing, using the stent, a EEG signal indicative of the electrical signal to processing circuitry configured to determine the electroencephalogram using the EEG signal.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example medical system and a stent.

FIG. 2 is a schematic view an example stent.

FIG. 3 is a schematic end view of the stent of FIG. 2 in a deployment configuration.

FIG. 4 is a schematic end view of the stent of FIG. 2 and FIG. 3 in an expanded configuration.

FIG. 5 illustrates an example technique for generating an EEG alert using processing circuitry.

FIG. 6 illustrates an example technique for generating an oxygen alert using the processing circuitry.

FIG. 7 illustrates another example technique for generating an oxygen alert using the processing circuitry.

FIG. 8A illustrates a technique for generating a flow alert using the processing circuitry.

FIG. 8B illustrates another technique for generating a flow alert using the processing circuitry.

FIG. 9 illustrates an example technique for generating a stroke alert using the processing circuitry.

FIG. 10 illustrates another example technique for generating a stroke alert using the processing circuitry.

FIG. 11 schematic illustration of a display device of the medical system.

FIG. 12 illustrates an example technique for monitoring a patient using the medical system.

### DETAILED DESCRIPTION

Stroke is a serious medical condition that can cause permanent neurological damage, complications, and death. Stroke may be characterized as the rapidly developing loss of brain functions due to a disturbance in the blood vessels supplying blood to the brain. The loss of brain functions can be a result of ischemia (lack of blood supply) caused by thrombosis or embolism, or hemorrhage (e.g., a ruptured blood vessel). During a stroke, the blood supply to an area of a brain may be decreased, which can lead to dysfunction of the brain tissue in that area.

Stroke is the number two cause of death worldwide and the number one cause of disability. Speed to treatment is the critical factor in stroke treatment as 1.9M neurons are lost per minute on average during stroke. Stroke diagnosis and time between event and therapy delivery are the primary barriers to improving therapy effectiveness. Stroke has various etiologies, including ischemic stroke (representing approximately 65% of all strokes), hemorrhagic stroke (representing approximately 10% of all strokes), and others (e.g., cryptogenic strokes). Strokes can be considered as having neurogenic and/or cardiogenic origins.

The disclosure provides a medical system configured to detect and/or monitor physiological parameters of a patient using a stent. In examples, the medical system is configured to monitor one or more physiological parameters of the patient including an electroencephalogram (EEG) of the patient, an oxygen level of the blood patient (e.g., a jugular venous oxygen saturation (SjVO2)), and/or a blood flow of the patient (e.g., a cerebral blood flow). The medical system may include processing circuitry configured to monitor the physiological parameters and compare the physiological parameters to one or more thresholds. The medical system may be configured to indicate that the patient may have experienced a stroke based on the comparison of the physiological parameters with the thresholds. In examples, the processing circuitry is configured to indicate a type of stroke the patient may have experienced based on the comparison. For example, the processing circuitry may be configured to indicate that the patient may have experienced an ischemic stroke, a hemorrhagic stroke, or another type of stroke based on the comparison. In some examples, the medical system is configured to indicate that the patient may have experienced a seizure (e.g., during an ablation such as a laser ablation, a legion surgery procedure, a tumor removal procedure, and/or other procedures).

The medical system includes a stent configured to be positioned (e.g., by a clinician) within an anatomical volume of the patient. In examples, the stent is configured to be positioned within a jugular vein of the patient. The stent is configured to monitor one or more of the EEG, the oxygen level, and the blood flow. The stent may be expandable from a deployment configuration to an expanded configuration, such that a clinician may intravenously transport the stent to the anatomical volume (e.g., the jugular vein) of the patient in the deployment configuration and, once positioned within the lumen, cause the stent to expand to the expanded configuration.

The stent is configured to monitor the one or more physiological parameters of the patient (e.g., one or more of the EEG, the oxygen level, and the blood flow) and provide one or signals indicative of the physiological parameters to the processing circuitry (e.g., when the stent is in the expanded configuration and positioned within the anatomical volume). The stent may be configured to contact a wall of the anatomical volume (e.g., a vessel wall of the jugular vein) when the stent is radially expanded from the deployment configuration to the expanded configuration. In examples, the stent defines a lumen (“stent lumen”) at least when the stent is in the expanded configuration. The stent may be configured such that a blood flow of the patient (e.g., blood flow within the jugular vein) flows through the stent lumen at least when the stent is in the expanded configuration. In examples, a proximal portion of the stent defines a proximal opening which opens to the stent lumen and a distal portion of the stent defines a distal opening which opens to the stent lumen. The stent may be configured such that the blood flow flows between the proximal opening and the distal opening via the stent lumen when the stent is in the expanded configuration and positioned within the anatomical volume of the patient. For example, in some examples, the stent may be configured to position within the anatomical volume such that the blood flows from the distal opening to the proximal opening. In some examples, the stent may be configured to position within the anatomical volume such that the blood flows from the proximal opening to the distal opening.

The stent includes a skeleton supporting one or more electrodes configured to receive (e.g., detect and/or sense) an electrical signal indicative of the EEG of the patient. The stent is configured to communicate an EEG signal indicative of the electrical signal from the one or more electrodes to the processing circuitry. In some examples, the stent is configured to conduct the EEG signal and/or electrical signal from the one or more electrodes to the processing circuitry. In some examples, the stent is configured to wirelessly communicate the EEG signal to the processing circuitry. In examples, the one or more electrodes are configured to detect an electrical signal indicative of the spontaneous bioelectric activity of the brain of the patient at least when the stent is positioned within the anatomical volume and the stent is in the expanded configuration. In some examples, the electrical signal is indicative of electrical activity of heart or other physiological functions of the patient.

For example, the one or more electrodes may be configured to detect electrical signals which include features representative of the bioelectric activity of the brain, such as amplitudes and/or frequencies in one or more frequency bands. The one or more electrodes may be configured to detect electrical signals in frequency bands such as delta (e.g., 0.5-4 Hz), theta (e.g., 4-7 Hz), alpha (e.g., from about 8-12 Hz, including an alpha1 band of about 8-10 Hz and an alpha2 band of about 10-12 Hz), beta (e.g. 13-30 Hz) and gamma (e.g., 30-40 Hz) bands. The one or more electrodes may include electrodes having any geometry, including button electrodes, ring electrodes, plate electrodes, substantially flat electrodes, substantially elongate electrodes, and/or other geometries. In some examples, the one or more electrodes comprise one or more conductive fibers, such as, for example, one or more conductive fibers including gold or silver. For example, the one or more conductive fibers comprise a conductive fabric supported by the skeleton. The stent may be configured such that the conductive fabric substantially surrounds at least some portion of a longitudinal axis extending through the stent lumen. In examples, the stent is configured such that the conductive fabric substantially defines a boundary of the stent lumen (e.g., a boundary surrounding the longitudinal axis).

The stent may be configured to determine the oxygen level of blood in the anatomical volume (e.g., a jugular vein) using a light emitter device. The light emitter device may be configured to project light into blood within the anatomical volume at least when the stent is in the expanded configuration and positioned within the anatomical volume. In examples, the stent (e.g., the skeleton) supports the light emitter device such that the light is projected at least within the stent lumen. In examples, the light emitter device is configured to illuminate blood within the stent lumen when the stent is in the expanded configuration and positioned within the anatomical volume. The light emitter device may be configured to illuminate blood and/or other tissue structures (e.g., a vessel wall of the anatomical volume). In examples, the light emitter device includes a waveguide (e.g., a fiber optic cable or other waveguide) configured to direct light from a light source. The light emitter device may include a lens (e.g., a Grinnel or other lens) optically coupled to the waveguide. The lens may be configured to receive light emitted by the light source and project the light to illuminate the blood and/or other tissue structures. In examples, the light source includes one or more laser devices configured to provide the light at one or more wavelengths to the waveguide.

The light emitter device is configured to project the light to cause a photoacoustic response of blood cells (e.g., hemoglobin) of the blood. The light emitter device may be configured to emit light such that blood cells absorb photon energy of one or more wavelengths of the light and cause an acoustic wave (e.g., a pressure wave) to propagate within the blood. For example, the blood cells may absorb photons and generate heat, inducing a thermal-elastic expansion that generates a local pressure rise and emits acoustic waves. The acoustic waves may be detected using, for example, a photoelectric sensor and processed using spectral-based methods to determine an oxygen level of the blood.

For example, the light emitter device may be configured to emit light (e.g., near-infrared light) at one or more wavelengths which cause hemoglobin in the blood to emit acoustic waves. The hemoglobin may include oxygenated hemoglobin (HbO2) and/or deoxygenated hemoglobin (HbR). The HbO2 and HbR molecules may be expected to have different wavelength-specific optical absorptions (e.g., due to oxygen binding and/or a substantial lack thereof). For example, light at a lower wavelength (e.g., between about 500-600 nm) may be more strongly absorbed by HbR as compared to HbO2. Light at a higher wavelength (e.g., about 700-900 nm) may be more strongly absorbed by HbO2 as compared to HbR. The light emitter device may be configured to illuminate the blood using light having at least one wavelength in order to provoke an acoustic response allowing for discrimination of the HbO2 and HbR. The medical system (e.g., the processing circuitry) may be configured to determine an oxygen level of the blood (e.g., an SjVO2) based on the acoustic response. In some examples, the light emitter device may be configured to illuminate the blood with light including a first wavelength (e.g., a wavelength less than 805 nm, such as about 700 nm) and a second wavelength greater than the first wavelength (e.g. a wavelength greater than or equal to 805 nm, such as a wavelength of about 1064 nm).

The acoustic response may be expected to cause an acoustic wave (e.g., a pressure wave) to propagate through the blood within the anatomical volume of the patient. The stent may include a first material configured to detect a first portion of the acoustic wave (“first acoustic wave portion”). In examples, the stent includes a second material configured to detect a second portion of the acoustic wave (“second acoustic wave portion”). In examples, the stent is configured such that the first material and/or the second material is in fluidic communication with blood within the anatomical volume at least when the stent is in the expanded configuration and the stent is positioned within the anatomical volume. The stent is configured such that interaction of the first material and the first acoustic wave portion causes the first material to develop a first electric potential (e.g., an electric potential relative to a reference potential). The stent may be configured such that interaction of the second material and the second acoustic wave portion causes the second material to develop a first electric potential (e.g., an electric potential relative to a reference potential).

For example, the first material may be supported by the stent such that a first mechanical stress develops within the first material when the first acoustic wave portion impacts the first material. The first material may be configured to develop the first electric potential in response to the first mechanical stress. The first material may be a piezoelectric material (e.g., poly vinylidene fluoride (PVF) or another piezoelectric material) configured to develop the first electric potential in response to the mechanical stress. The first material may be configured such that a first electrical parameter (e.g., the first electric potential, a first impedance of the first material, and/or another electrical parameter) develops and/or is altered based the interaction between the first material and the first acoustic wave portion (e.g., based on the mechanical stress caused by the interaction between the first material and the first acoustic wave portion). The stent is configured to communicate a first signal indicative of the first electrical parameter and/or the alteration thereof from the first material to the processing circuitry.

The first material may have any geometry, including a button-shape, a ring electrodes, a plate, a substantially elongate shape, and/or other geometries. In some examples, the first material comprise one or more first elongate fibers. In examples, the first elongate fiber is a conductive fiber. In some examples the first elongate fiber is substantially supported by the skeleton and/or the conductive fabric comprising the one or more electrodes. For example, the first elongate fiber may be substantially woven into the conductive fabric. In some examples, the stent is configured such that the first material substantially surrounds at least some portion of the longitudinal axis extending through the stent lumen.

The stent may be configured such that interaction of the second material and the second acoustic wave portion causes the second material to develop the second electric potential (e.g., a second electric potential relative to a reference potential). For example, the second material may be supported by the stent such that a second mechanical stress develops within the second material when the second acoustic wave portion impacts the second material. The second material may be configured to develop the second electric potential in response to the second mechanical stress. In examples, the second material is a second piezoelectric material (e.g., poly vinylidene fluoride (PVF) or another piezoelectric material) configured to develop the second electric potential in response to the second mechanical stress. The second material may be configured such that a second electrical parameter (e.g., the second electric potential, a second impedance, and/or another electrical parameter) develops and/or is altered based the interaction between the second material and the second acoustic wave portion. The stent is configured to communicate a second signal indicative of the second electrical parameter developed and/or the alteration thereof from the second material to the processing circuitry.

The second material may have any geometry, including a button-shape, a ring electrodes, a plate, a substantially elongate shape, and/or other geometries. In some examples, the second material is supported by the light emitter device. For example, the second material may be supported by the light emitter device in substantial proximity to the lens and/or waveguide. In some examples, the second material may comprise one or more second elongate fibers substantially supported by the skeleton and/or the conductive fabric comprising the one or more electrodes. For example, the second elongate fiber may be substantially woven into the conductive fabric. In some examples, the stent is configured such that the second material substantially surrounds at least some portion of the longitudinal axis extending through the stent lumen.

The processing circuitry is configured to determine an oxygen level of the blood within the anatomical volume using an oxygen signal provided by the stent, wherein the oxygen signal is one of the first signal or the second signal. In examples, the processing circuitry conducts a spectral analysis of the oxygen signal to determine the oxygen level. For example, the processing circuitry may conduct the spectral analysis to determine an SjV02 of the blood within the anatomical volume (e.g., within the jugular vein).

The processing circuitry may be configured to determine a flow rate of the blood within the anatomical volume using a flow signal provided by the stent. In some examples, the flow signal is the other of first signal or the second signal. In some examples, the flow signal is another signal provided by the stent. The flow signal may be indicative of a flow rate through the stent lumen. In some examples, the processing circuitry is configured to determine one or more photoacoustic images using the flow signal. The processing circuitry may be configured to determine the blood flow rate using the one or more photoacoustic images (e.g., by photoacoustic vector tomography or another method configured to map vectors of a blood flow). In some examples, the stent may be configured to sense and/or provide the flow signal in other ways.

For example, the second material may be configured to receive light scattered and/or reflected by moving objects in the blood flow such as such as red blood cells. The processing circuitry may be configured to determine a doppler flux based on comparison of the light scattered and/or reflected with the light emitted by the light emitter device (e.g., based on a change in frequency). The processing circuitry may be configured to determine the blood flow rate using the doppler flux. The processing circuitry may be configured to determine the flow rate using the flow signal in other ways in other examples.

As used herein, the first acoustic wave portion and the second acoustic wave portion may refer to portions of a single acoustic wave caused by the light emitter device or may refer to substantially separate acoustic waves caused by light emitter device. For example, the light emitter device may be configured to emit light having first optical characteristics (e.g., a first wavelength range, a first pulse rate, a first power, and/or other first characteristics) to cause a first acoustic wave. The first acoustic wave portion and the second acoustic wave portion may refer to portions of the first acoustic wave, such that the first material interacts with a first portion of the first wave and the second material interacts with a second portion of the first acoustic wave. In other examples, the first acoustic wave portion may refer to a portion of the first acoustic wave, and the second acoustic wave portion may refer to a portion of a second acoustic wave different from the first acoustic wave. For example, the light emitter device may be configured to emit light having the first optical characteristics to cause the first acoustic wave and emit light having second optical characteristics (e.g., a second wavelength range, a second pulse rate, a second power, and/or other second characteristics) different from the first optical characteristics to cause the second acoustic wave. Hence, in some examples, when the first material interacts with the first acoustic wave portion and the second material interacts with the second acoustic wave portion, this may refer to the first material and the second material interacting with different portions of an acoustic wave caused by a light emission having the first optical characteristics. In other examples, when the first material interacts with the first acoustic wave portion and the second material interacts with the second acoustic wave portion, this may refer to the first material interacting with a portion of the first acoustic wave and the second material interacting with a portion of the second acoustic wave.

The processing circuitry is configured to determine an EEG parameter using the EEG signal received from the stent. The processing circuitry may be configured to determine an oxygen parameter using the oxygen signal received from the stent (e.g., one of the first signal or the second signa), and may be configured to determine a flow parameter using the flow signal received from the stent (e.g., the other of the first signal or the second signal). The processing circuitry may be configured to compare the EEG parameter to an EEG threshold and determine when the EEG parameter satisfies a criteria based on the EEG threshold (e.g., when the EEG parameter is less than, greater than, or equal to the EEG threshold). The processing circuitry may be configured to compare the oxygen parameter to an oxygen threshold and determine when the oxygen parameter satisfies a criteria based on oxygen threshold (e.g., when the oxygen parameter is less than, greater than, or equal to the oxygen threshold). The processing circuitry may be configured to compare the flow parameter to a flow threshold and determine when the flow parameter satisfies a criteria based on flow threshold (e.g., when the flow parameter is less than, greater than, or equal to the flow threshold). The processing circuitry may be configured to indicate that a patient may have experienced a stroke based on one or more of the comparison of the EEG parameter and the EEG threshold, the comparison of the oxygen parameter and the oxygen threshold, and the comparison of the flow parameter and the flow threshold. In examples, the processing circuitry is configured to indicate a type of stroke the patient may have experienced based on the comparison. For example, the processing circuitry may be configured to indicate that the patient may have experienced an ischemic stroke, a hemorrhagic stroke, or another type of stroke based on the comparison.

The processing circuitry may be configured to determine (e.g., establish) one or more of the EEG threshold, the oxygen threshold, and the flow threshold. For example, medical system may be configured such that the stent provides one or more of the EEG signal, the oxygen signal, and the flow signal to the processing circuitry over an initial period of time (e.g., a period of time when the patient is considered medically stable). The processing circuitry may determine the EEG threshold using the EEG signals provided over the initial period, determine the oxygen threshold using the oxygen signals provided over the initial period, and determine the flow threshold using the flow signals provided over the initial period. Hence, the EEG threshold, the oxygen threshold, and the flow threshold may be indicative of parameters expected and determined when the patient is in the medically stable state.

The processing circuitry may be configured to receive one of more of the EEG signal, the oxygen signal, and the flow signal subsequent to the initial period of time to determine one or more of the EEG parameter, the oxygen parameter, and the flow parameter. The processing circuitry may compare the subsequently determined EEG parameter, oxygen parameter, and flow parameter to the EEG threshold, the oxygen threshold, and the flow threshold to substantially monitor for parameter changes which might be indicative of a stroke event. The processing circuitry may be configured to provide a stroke alert (e.g., visual, audible, or other alert) to a clinician when a combination of the comparisons with the thresholds may be indicative of a stroke event.

For example, the processing circuitry may be configured to initiate an EEG alert when the EEG parameter satisfies a criteria based on the EEG threshold. The processing circuitry may be configured to initiate an oxygen alert when the oxygen parameter satisfies a criteria based on the oxygen threshold. The processing circuitry may be configured to initiate a flow alert when the flow parameter satisfies a criteria based on the flow threshold. The processing circuitry may be configured to provide the stroke alert when the processing circuitry initiates some combination of the EEG alert, the oxygen alert, and the flow alert during a monitoring period. In some examples, the processing circuitry is configured to provide the stroke alert when the processing circuitry initiates all of the EEG alert, the oxygen alert, and the flow alert during the monitoring period.

The processing circuitry may be configured to determine the EEG threshold using a Delta-to-Alpha ratio (“DAR”) determined using the EEG signals received over the initial period of time. In some examples, the processing circuitry determines the EEG threshold by determining a change in the DAR over the initial period of time. In some examples, the processing circuitry determines the EEG threshold by determining a time rate of change of the change in the DAR. Hence, the EEG threshold may be indicative of a change in the DAR or a time rate of change which might be expected with the patient in a medically stable state. The processing circuitry may determine the EEG parameter by determining a change in the DAR subsequent to the initial period of time, and/or by determining a time rate of change of the change in the DAR subsequent to the initial period of time. Hence, the comparison of the EEG parameter with the EEG threshold may be indicative of a DAR change and/or a time rate of change that could signal a stroke event has occurred.

The processing circuitry may be configured to determine the oxygen threshold using an SjVO2 determined using the oxygen signals received over the initial period of time. In some examples, the processing circuitry determines the oxygen threshold by determining a change in the SjVO2 over the initial period of time. In some examples, the processing circuitry determines the oxygen threshold by determining a time rate of change of the SjVO2. Hence, the oxygen threshold may be indicative of a change in the SjVO2 or a time rate of change of the SJVO2 which might be expected with the patient in a medically stable state. The processing circuitry may determine the oxygen parameter by determining a change in the SjVO2 subsequent to the initial period of time, and/or by determining a time rate of change of the change in the SjVO2 subsequent to the initial period of time. Hence, the comparison of the oxygen parameter with the oxygen threshold may be indicative of a SjVO2 change and/or a time rate of change that could signal a stroke event has occurred.

The processing circuitry may be configured to determine the flow threshold using the flow signals received over the initial period of time, such that the flow threshold is indicative of a blood flow rate which might be expected with the patient in a medically stable state. The processing circuitry may determine the flow parameter using flow signals received subsequent to the initial period of time. Hence, the comparison of the flow parameter with the flow threshold may be indicative of a change in the blood flow rate that could signal a stroke event has occurred. In examples, the processing circuitry is configured to distinguish between an Ischemic stroke and a hemorrhagic stroke based on the comparison of the flow parameter and the flow threshold.

The stent thus allows for monitoring and/or sensing an EEG, an oxygen level, and a flow rate using a single stent placed in an anatomical volume of the patient, as opposed to methodologies which may require coordinated use of several substantially separate systems. The stent may be configured for positioning within a jugular vein of the patient, such that changes in at least the oxygen level and flow rate may be indicative of a stroke event, as opposed to systems which may be configured to monitor for oxygen level and flow rate in other portions of the patient. The processing circuitry allows for determining and/or assessment of changes to the EEG, the oxygen level, and the flow rate from threshold parameters determined by the processing circuitry. The processing circuitry allows specific classification of stroke events (e.g., ischemic, hemorrhagic) based the changes of the EEG, the oxygen level, and the flow rate from the EEG thresholds, the oxygen level threshold, and the flow thresholds determined by the processing circuitry.

Hence, the disclosure provides a medical system including a stent configured to be positioned in an anatomical volume of a patient, such as a jugular vein of a patient. The stent is configured to sense an EEG signal indicative of an EEG of the patient and provide the EEG signal to processing circuitry. In examples, the medical system senses the EEG signal using one or more electrodes of the stent. The stent is configured to sense an oxygen signal indicative of an oxygen level of the patient and provide the oxygen signal to the processing circuitry. In examples, the medical system senses the oxygen signal using a first material of the stent. The stent is configured to sense a flow signal indicative of a blood flow rate of the patient and provide the flow signal to the processing circuitry. In examples, the medical system sense the flow signal using a second material of the stent. The processing circuitry may be configured to compare the EEG parameter with an EEG threshold, compare the oxygen parameter with an oxygen threshold, and compare the flow parameter with a flow threshold. The processing circuitry may be configured to provide a stroke alert when the processing circuitry initiates some combination of or all of an EEG alert, an oxygen alert, and an flow alert based on the comparisons.

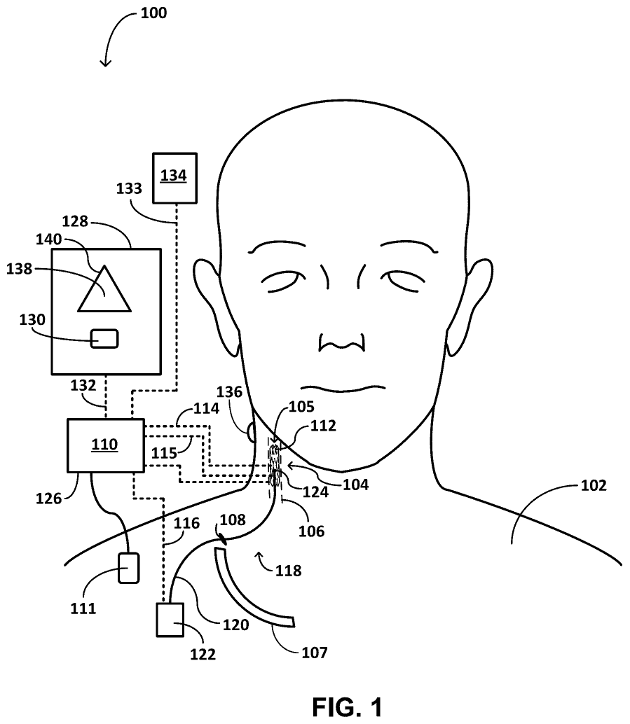

FIG. 1 is a conceptual diagram illustrating an example medical system 100 configured to monitor physiological parameters of a patient 102 using a stent 104. Stent 104 is configured to be positioned within an anatomical volume of patient 102, such as anatomical volume 106 of patient 102. Anatomical volume 106 may be an anatomical volume defined by, for example, a jugular vein (e.g., an external jugular vein, an internal jugular vein, an anterior jugular vein), a carotid artery, or another anatomical structure of patient 102. Stent 104 may be configured to expand from a deployment configuration to an expanded configuration, such that a clinician may intravenously transport stent 104 to anatomical volume 106 in the deployment configuration and, once positioned within anatomical volume 106, cause stent 104 to expand to the expanded configuration. In examples, medical system 100 may include a delivery system 107 configured to deliver stent 104 (e.g., in the deployment configuration) to anatomical volume 106 and/or another anatomical volume of patient 102. For example, stent 104 may be advanced to anatomical volume 106 or another anatomical volume through a substantially tubular member of the delivery system, such as a sheath or guide catheter. The tubular member may be placed with its distal end in anatomical volume 106 before stent 104 is advanced through the surrounding tubular member. Stent 104 and/or delivery system 107 may be configured such that the clinician may cause stent 104 to radially expand from the deployment configuration to the expanded configuration when stent 104 is positioned within anatomical volume 106. Stent 104 may be configured such that delivery system 107 may deliver and/or retrieve stent 104 to anatomical volume 106 intravascularly, such as via an access 108 or another access.

Medical system 100 may be configured to sense physiological parameters of patient 102 including an EEG of patient 102, an oxygen level of the blood of patient 102 (e.g., an SjVO2 of blood within anatomical volume 106 (e.g., within a jugular vein)), and/or a blood flow of the patient 102 (e.g., a cerebral blood flow within anatomical volume 106 (e.g., within the jugular vein)). Medical system 100 is configured to sense the physiological parameters using stent 104. Medical system 100 includes processing circuitry 110 configured to monitor the physiological parameters and compare the physiological parameters to one or more thresholds. Processing circuitry 110 is configured to indicate that patient 102 may have experienced a stroke (e.g., an ischemic stroke, a hemorrhagic stroke, or another type of stroke) based on the comparison of the physiological parameters and the thresholds.

Stent 104 is configured to monitor the one or more physiological parameters (e.g., one or more of the EEG, the oxygen level, and the blood flow) and provide one or signals indicative of the physiological parameters to processing circuitry 110 (e.g., when stent 104 is in the expanded configuration and positioned within anatomical volume 106 or another anatomical volume). In examples, stent 104 defines a stent lumen 105 at least when stent 104 is in the expanded configuration. Stent 104 may be configured such that a blood flow of patient 102 (e.g., blood flow within anatomical volume 106) flows through stent lumen 105 when stent 104 is positioned (e.g., by a clinician) within anatomical volume 106.

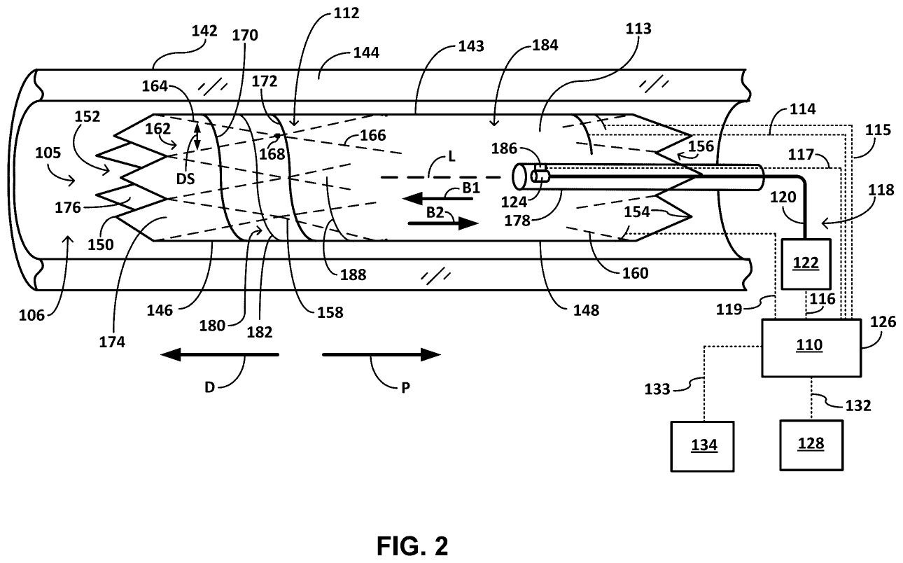

Stent 104 includes a skeleton 112 supporting one or more electrodes (e.g., electrode 113 (FIG. 2)). The one or more electrodes are configured to receive (e.g., detect and/or sense) an electrical signal indicative of the EEG of patient 102 when stent 104 is positioned within anatomical volume 106. Stent 104 (e.g., the one or more electrodes) may be configured to detect an electrical signal indicative of the spontaneous bioelectric activity of the brain of patient 102 at least when stent 104 is positioned within anatomical volume 106 and stent 104 is in the expanded configuration. In some examples, Stent 104 (e.g., the one or more electrodes) is configured to detect an electrical signal indicative of electrical activity of the heart of patient 102 or other physiological functions of patient 102.

Stent 104 (e.g., the one or more electrodes) may be configured to detect electrical signals representative of the bioelectric activity of the brain of patient 102 in one or more frequency bands, such as delta (e.g., 0.5-4 Hz), theta (e.g., 4-7 Hz), alpha (e.g., from about 8-12 Hz, including an alpha1 band of about 8-10 Hz and an alpha2 band of about 10-12 Hz), beta (e.g. 13-30 Hz) and gamma (e.g., 30-40 Hz) bands. The one or more electrodes may include electrodes having any geometry, however, in some examples, the one or more electrodes comprise one or more conductive fibers. In some examples, the one or more conductive fibers comprise a conductive fabric supported by skeleton 112. In some examples, skeleton 112 supports the conductive fabric such that the conductive fabric substantially defines a boundary of stent lumen 105.

Stent 104 is configured to communicate an EEG signal indicative of the electrical signals sensed by the one or more electrodes to processing circuitry 110. For example, stent 104 may be configured to communicate the EEG signal to processing circuitry 110 via comm link 114. Processing circuitry 110 is configured to determine an EEG of patient 102 using the EEG signal. In examples, processing circuitry 110 is configured to determine the EEG over at least the delta, theta, alpha, beta, and/or and gamma bands.

Stent 104 may be configured to sense the oxygen level of blood in anatomical volume 106 using a light emitter device 118. Light emitter device 118 is configured to project light into blood within anatomical volume 106 at least when stent 104 is positioned within anatomical volume 106. Stent 104 (e.g., skeleton 112) may support light emitter device 118 such that the light is projected at least within stent lumen 105. In examples, light emitter device 118 includes one or more waveguides 120 (“waveguides 120”) (e.g., a fiber optic cable or other waveguide) and a light source 122. Waveguides 120 may be configured to direct light from light source 122 to cause light emitter device 118 to project the light within stent lumen 105. In examples, light emitter device 118 includes a lens 124 (e.g., a Grinnel or other lens) optically coupled to waveguides 120. Lens 124 may be configured to receive light emitted by light source 122 via waveguides 120 and project the light to illuminate blood within stent lumen 105. In examples, light source 122 includes one or more laser devices configured to provide the light at one or more wavelengths (e.g., at least a first wavelength less than 805 nm and a second wavelength greater than 805 nm) to waveguides 120.

In examples, the laser may be a short duration pulsed laser. In some examples, light source 122 (e.g., the laser) is configured to emit light pulses having a pulse width less than about 500 nanoseconds, in some examples less than 250 nanoseconds, and in some examples less than 100 nanoseconds. In some examples, light source 122 (e.g., the laser) may emit light pulses at a period of less than 0.1 seconds, in some examples less than 0.05 seconds. Stent 104 may be configured to detect an acoustic wave caused by a light pulse emitted by light source 122. For example, stent 104 may be configured to detect a first acoustic wave caused by a first light pulse, a second acoustic wave caused by a second light pulse emitted subsequent to the second light pulse, a third acoustic wave caused by a third light pulse emitted subsequent to the second light pulse, and so on. Stent 104 may be configured to provide a first flow signal and/or first oxygen signal resulting from the first acoustic wave to processing circuitry 110, provide a second flow signal and/or second oxygen signal resulting from the second acoustic wave to processing circuitry 110, provide a third flow signal and/or third oxygen signal resulting from the third acoustic wave to processing circuitry 110, and/or provide further flow signals and/or further oxygen signals resulting from further acoustic waves caused by light source 122 to processing circuitry 110. Processing circuitry 110 may be configured to determine a first oxygen parameter and/or first oxygen threshold using the first oxygen signal, determine a first flow parameter and/or first flow threshold using the first flow signal, determine a second oxygen parameter and/or second oxygen threshold using the second oxygen signal, determine a second flow parameter and/or second flow threshold using the second flow signal, determine a third oxygen parameter and/or third oxygen threshold using the third oxygen signal, determine a third flow parameter and/or third flow threshold using the third flow signal, and so on. In some examples, stent 104 is configured to provide and processing circuitry 110 is configured to receive an oxygen signal and/or a flow signal at least 10 times per second, in some examples at least 20 times per second.

Light emitter device 118 is configured to project the light to cause a photoacoustic response of blood cells (e.g., hemoglobin) of blood within anatomical volume 106. Light emitter device 118 is configured to emit the light to cause the blood cells to absorb photon energy of one or more wavelengths of the light and cause an acoustic wave (e.g., a pressure wave) to propagate within the blood. In some examples, light emitter device 118 is configured to emit light at one or more wavelengths which cause hemoglobin in the blood to absorb photons and emit acoustic waves. For example, light emitter device 118 may be configured to emit light at one or more wavelengths causing oxygenated hemoglobin (HbO2) and/or deoxygenated hemoglobin (HbR) to emit acoustic waves. In examples, light emitter device 118 is configured to emit light at a first wavelength (e.g., a wavelength less than 805 nm, such as about 700 nm) and at a second wavelength greater than the first wavelength (e.g. a wavelength greater than or equal to 805 nm, such as a wavelength of about 1064 nm) to allow for discrimination between acoustic waves caused by HbO2 and Hbr. For example, light emitter device 118 may be configured to emit one or more pulses of light at the first wavelength to provoke an acoustic wave caused largely by photon absorption of HbO2. Light emitter device 118 may be configured to emit one or more pulses of light at the second wavelength (e.g., separate from the emission at the first wavelength) to provoke an acoustic wave caused largely by photon absorption of Hbr.

Stent 104 is configured to detect at least a portion of the acoustic wave using a material configured to interact with the portion of the acoustic wave (e.g., one of first material 180 or second material 184 (FIG. 2)). Stent 104 may be configured such that the material is in fluidic communication with blood within anatomical volume 106 when stent 104 is positioned within anatomical volume 106. In examples, stent 104 is configured such that interaction of the material and the acoustic wave causes the material to develop an electric potential. The material may be configured such that an electrical parameter of the material (e.g., the electric potential, an impedance of the material, and/or another electrical parameter) develops and/or is altered based the interaction between the material and the acoustic wave. In some examples, skeleton 112 substantially supports the material. For example, the material may be one or more elongate fibers substantially woven into a conductive fabric defining the one or more electrodes. In some examples, light emitter device 118 (e.g., a housing supporting and/or surrounding waveguides 120) supports the material. For example, the light emitter device 118 may support the material in proximity to a distal end of one of waveguides 120.

In some examples, stent 104 may be configured to sense the oxygen level of blood in anatomical volume 106 based on a reflectance of the emitted light. For examples, light emitter device 118 may be configured to gather some portion of the emitted light reflected by the blood within anatomical volume 106 (e.g., reflected by blood cells within anatomical volume 106). Light emitter device 118 may be configured to provide a reflected light signal indicative of the light gathered to processing circuitry 110. For example, in some examples, light emitter device 118 is configured to emit light into anatomical volume 106 (e.g., by emitting light into stent lumen 105) using a first waveguide of waveguides 120. Light emitter device 118 may be configured to provide the reflected light signal to processing circuitry 110 using a second waveguide of waveguides 120. Processing circuitry 110 may be configured to determine the oxygen level using the reflected light signal. In some examples, processing circuitry 110 is configured to assess an absorption of the emitted light by the blood in anatomical volume 106 and/or surrounding structures based on a comparison of the emitted light (e.g., provided to stent 104 via the first waveguide) and the reflected light signal (e.g., received from stent 104 via the second waveguide). Processing circuitry 110 may be configured to determine the oxygen level based on the comparison of the emitted light and the reflected light signal.

Stent 104 is configured to communicate an oxygen signal indicative of the electrical parameter developed and/or the alteration thereof from the material to processing circuitry 110. For example, stent 104 may be configured to communicate the oxygen signal to processing circuitry 110 via comm link 115. Processing circuitry 110 may be configured to determine an oxygen level of the blood within anatomical volume 106 using the oxygen signal. In examples, processing circuitry 110 is configured to determine an SjV02 of the blood within anatomical volume 106 (e.g., when anatomical volume 106 is a jugular vein).

In examples, processing circuitry 110 is configured to communicate with light emitter device 118 to cause light emitter device 118 to emit the light. For example, processing circuitry 110 may be configured to communicate with light emitter device 118 via comm link 116. In examples, processing circuitry 110 is configured to cause light emitter device 118 to emit light having specific optical characteristics, such as a specific wavelength range, a specific pulse rate, a specific power, and/or other optical characteristics. In some examples, processing circuitry 110 may be configured to cause light emitter device 118 emit light having first optical characteristics (e.g., having a first wavelength) to cause a first acoustic wave, such that stent 104 provides a first oxygen signal in response to the first acoustic wave. Processing circuitry 110 may be configured to (e.g., subsequently) cause light emitter device 118 emit light having second optical characteristics (e.g., having a second wavelength) to cause a second acoustic wave, such that stent 104 provides a second oxygen signal in response to the second acoustic wave. Processing circuitry 110 may be configured to determine the oxygen level using the first oxygen signal and the second oxygen signal.

Processing circuitry 110 may be supported (e.g., mechanically supported) by a housing 126. In some example, housing 126 may be configured to be implanted (e.g., subcutaneously implanted) within patient 102. In some example, housing 126 may be configured to be extracorporeal to patient 102 when, for example, stent 104 is intracorporeal to patient 102. Housing 126 may further support (e.g., mechanically support) some portion of light emitter device 118, such as, for example, light source 122 and/or portions of waveguides 120.

Stent 104 is configured to sense a flow signal indicative of a flow rate of the blood in anatomical volume 106. In examples, the flow signal is indicative of a flow rate through stent lumen 105. In some examples, stent 104 is configured to sense the flow signal using the light emitted by light emitter device 118 (e.g., the same light transmitted to provoke an acoustic wave sensed to provide the oxygen signal, or another emitted light). Stent 104 may be configured to communicate the flow signal to processing circuitry 110 (e.g., via a comm link 117). Processing circuitry 110 may be configured to determine a flow rate indicative of the blood flow within anatomical volume 106 using the flow signal. In examples, processing circuitry 110 conducts a spectral analysis of the flow signal to determine the flow rate.

In examples, stent 104 includes an additional material (e.g., the other of first material 180 or second material 184) configured to sense the flow signal. Stent 104 may be configured such that the additional material is in fluidic communication with blood within anatomical volume 106 when stent 104 is positioned within anatomical volume 106. In examples, stent 104 is configured such that interaction of the additional material and at least some portion of an acoustic wave caused by light emitter device 118 causes the additional material to develop an electric potential. The additional material may be configured such that an electrical parameter of the additional material (e.g., the electric potential, an impedance of the additional material, and/or another electrical parameter of the additional material) develops and/or is altered based the interaction between the additional material and the portion of the acoustic wave. In some examples, skeleton 112 substantially supports the additional material. For example, the additional material may be one or more elongate fibers substantially woven into a conductive fabric defining the one or more electrodes. In some examples, light emitter device 118 (e.g., a housing supporting and/or surrounding waveguides 120) supports the additional material. For example, the light emitter device 118 may support the additional material in proximity to a distal end of one of waveguides 120.

Stent 104 is configured to communicate a flow signal indicative of the electrical parameter developed and/or the alteration thereof from the additional material to processing circuitry 110. In examples, processing circuitry 110 is configured to determine one or more photoacoustic images using the flow signal. In some examples, processing circuitry 110 is configured to determine the blood flow rate using the one or more photoacoustic images (e.g., by photoacoustic vector tomography or another method configured to map vectors of a blood flow). In some examples, the additional material may be configured to receive light scattered and/or reflected by moving objects in the blood flow such as such as red blood cells. Processing circuitry 110 may be configured to determine a doppler flux based on comparison of the light scattered and/or reflected with the light emitted by light emitter device 118. Processing circuitry 110 may be configured to determine the blood flow rate using the doppler flux. In some examples, for example when skeleton 112 supports a conductive fabric, stent 104 may be configured to provide a flow signal indicative of an impedance and/or change in the impedance of the conductive fabric (e.g., due to interaction with an acoustic wave). Processing circuitry 110 may be configured to determine the blood flow rate using the flow signal indicative of the impedance and/or change in the impedance. Stent 104 may be configured to sense and provide the flow signal, and processing circuitry 110 may be configured to determine the flow rate of blood, in other manners in other examples.

Processing circuitry 110 is configured to determine an EEG parameter using the EEG signal received from stent 104. Processing circuitry 110 may be configured to determine an oxygen parameter using the oxygen signal received from stent 104. In examples, processing circuitry 110 is configured to determine a flow parameter using the flow signal received from stent 104. In some examples, medical system 100 includes a reference electrode 111 configured to provide a reference signal (e.g., a reference voltage) to processing circuitry 110. In examples, reference electrode 111 is configured to establish electrical communication with some portion of patient 102. Reference electrode 111 may be, for example, a skin electrode or other type of electrode. In other examples, reference electrode 111 may be a portion of processing circuitry 110. For example, reference electrode 111 may be an electrode configured to provide a reference voltage for use by other portions of processing circuitry 110. In some examples, processing circuitry 110 is configured to determine one or more of the EEG parameter, the oxygen parameter, and/or the flow parameter using the reference signal.

Processing circuitry 110 may be configured to compare the EEG parameter to an EEG threshold, compare the oxygen parameter to an oxygen threshold, and/or compare the flow parameter to a flow threshold. Processing circuitry 110 may be configured to indicate that patient 102 may have experienced a stroke based on one or more of the comparison of the EEG parameter and the EEG threshold, the comparison of the oxygen parameter and the oxygen threshold, and the comparison of the flow parameter and the flow threshold. In examples, processing circuitry 110 is configured to indicate a type of stroke the patient may have experienced based on the comparison. For example, processing circuitry 110 may be configured to indicate that the patient may have experienced an ischemic stroke, a hemorrhagic stroke, or another type of stroke based on the comparison.

In examples, processing circuitry 110 is configured to determine (e.g., establish) one or more of the EEG threshold, the oxygen threshold, and the flow threshold. For example, processing circuitry 110 may be configured to determine the EEG threshold (e.g., using the EEG signal), the oxygen threshold (e.g., using the oxygen signal), and the flow threshold (e.g., using the flow signal) during an initial period of time when patient 102 is considered (e.g., by a clinician) to be in a medically stable state. Processing circuitry 110 may be configured to subsequently receive one of more of the EEG signal, the oxygen signal, and the flow signal to determine one or more of the EEG parameter, the oxygen parameter, and the flow parameter. Processing circuitry 110 may compare the subsequently determined EEG parameter, oxygen parameter, and flow parameter to the EEG threshold, the oxygen threshold, and the flow threshold to substantially monitor for parameter changes which might indicate that patient 102 has experienced a stroke event.

For example, processing circuitry 110 may be configured to initiate an EEG alert when the EEG parameter satisfies a criteria based on the EEG threshold. Processing circuitry 110 may be configured to initiate an oxygen alert when the oxygen parameter satisfies a criteria based on the oxygen threshold. Processing circuitry 110 may be configured to initiate a flow alert when the flow parameter satisfies a criteria based on the flow threshold. Processing circuitry may be configured to provide the stroke alert when processing circuitry 110 initiates one or more of the EEG alert, the oxygen alert, and the flow alert during a monitoring period. In some examples, processing circuitry 110 is configured to provide the stroke alert when the processing circuitry initiates all of the EEG alert, the oxygen alert, and the flow alert during the monitoring period.

In some examples, processing circuitry 110 is configured to determine a metabolic rate (e.g., a cerebral metabolic rate of oxygen (CMRO2)) using one or more of the flow signal, flow parameter, oxygen signal, and/or oxygen parameter. The metabolic rate determined may be indicative of a rate of oxygen consumption by the brain of patient 102. In examples, the oxygen level determined by processing circuitry 110 using the oxygen parameter includes an SjVO2, and processing circuitry 110 is configured to determine the metabolic rate using the SjVO2 and one or more of the flow signal and/or flow parameter. In some examples, medical system 100 is configured to determine a reference oxygen level of patient 102 (e.g., using an oxygen sensor 136), and processing circuitry 110 is configured to determine the metabolic rate based on a comparison of the oxygen signal and/or oxygen parameter and the reference oxygen level. For example (e.g., when stent 104 is positioned in a jugular vein of patient 102), medical system 100 (e.g., oxygen sensor 136) may be configured to determine an oxygen level of blood in a vein or artery providing blood to the brain of patient 102 (e.g., a carotid artery of patient 102). Processing circuitry 110 may be configured to use the oxygen level of the blood in the vein or artery providing the blood to the brain of patient 102 as the reference oxygen level. In some examples (e.g., when stent 104 is positioned in a carotid artery of patient 102), medical system 100 (e.g., oxygen sensor 136) may be configured to determine an oxygen level of blood in a vein or artery receiving blood from the brain of patient 102 (e.g., a jugular vein of patient 102). Processing circuitry 110 may be configured to use the oxygen level of the blood in the vein or artery receiving the blood from the brain of patient 102 as the reference oxygen level.

In examples, medical system 100 includes a display device 128 configured to receive the stroke alert from processing circuitry 110. Display device 128 may be configured to provide an indication (e.g., a visual and/or audible indication) to a clinician when processing circuitry 110 has provided the stroke alert. For example, display device 128 may include an alarm device 130 configured to provide a visual and/or audible alarm when processing circuitry 110 provides the stroke alert. In examples, processing circuitry 110 is configured to communicate the stroke alert to display device 128 using a comm link 132.

In some examples, medical system 100 (e.g., display device 128) includes a visual display 138 configured to provide a visual indication indicative of one or more or the EEG parameter, the oxygen parameter, the flow parameter, and/or the metabolic rate. Visual display 138 may be configured such that the visual indication of the one or more or the EEG parameter, the oxygen parameter, the flow parameter, and/or the metabolic rate provides a indication of the neurovascular coupling of patient 102. In examples, display device 128 is configured to receive an flow input signal indicative of the flow parameter from processing circuitry 110, receive an oxygen input signal indicative of the oxygen parameter from processing circuitry 110, and/or receive a metabolic rate input indicative of the metabolic rate from processing circuitry 110. Visual display 138 may be configured to display a flow visual indication indicative of the flow input signal, display an oxygen visual indication indicative of the oxygen input signal, and/or display a metabolic rate visual indication indicative of the metabolic rate input signal. In some example, visual display 138 configured to provide the flow visual indication, the oxygen visual indication, and the metabolic rate visual indication as a point on a triangle graph 140. In examples, triangle graph 140 defines a first vertex corresponding to the flow input signal, a second vertex corresponding to the oxygen input signal, and a third vertex corresponding to the metabolic rate input signal. In examples, display device 128 is configured to be extracorporeal to patient 102 when, for example, stent 104 is intracorporeal to patient 102.

Although the examples herein discuss delivery, positioning, and/or retrieval of stent 104 largely within a jugular vein of patient 102, medical system 100 may be configured to deliver, position, and/or retrieve stent 104 in other anatomical volumes of patient 102 and provide at least one of an EEG signal, an oxygen signal, a flow signal, or another signal indicative of a physiological characteristic of patient 102 (e.g., a single indicative of an electrocardiogram of patient 102) in a like manner as that described for a jugular vein of patient 102.

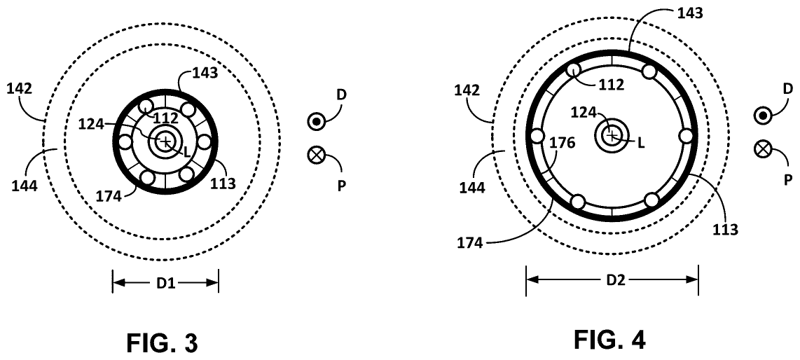

FIG. 2 is a schematic plan view of medical system 100 including a stent 104 in an expanded configuration, with a distal direction D and a proximal direction P parallel to the page. A representative anatomical vessel 142 (e.g., a jugular vein) and a representative vessel wall 144 defining anatomical volume 106 is depicted with stent 104 in FIG. 2 for context. FIG. 3 is a schematic end view of stent 104 in a deployment configuration, with distal direction D proceeding out of the page and proximal direction P proceeding into the page. FIG. 4 is a schematic end view of stent 104 in the expanded configuration, with distal direction D proceeding out of the page and proximal direction P proceeding into the page. FIG. 3 and FIG. 4 depict vessel 142 and vessel wall 144 in dashed lines for reference.

In examples, an outer surface 143 of stent 104 is configured to define a first cross-sectional dimension D1 (e.g., a first diameter) perpendicular to a longitudinal axis L defined by stent 104 when stent 104 is in the deployment configuration of FIG. 3. Outer surface 143 may be configured to define a second cross-sectional dimension D2 (e.g., a second diameter) perpendicular to longitudinal axis L and less than first cross-sectional diameter D1 when stent 104 is in the expanded configuration of FIG. 4. In examples, stent 104 is configured to transition from the deployment configuration to the expanded configuration (e.g., when stent 104 is positioned within anatomical volume 106). Stent 104 may be configured to transition from the expanded configuration to the deployment configuration (e.g., when stent 104 is positioned within anatomical volume 106). In examples, stent 104 is configured such that outer surface 143 radially displaces outward in a direction way from longitudinal axis L when stent 104 transitions from the deployment configuration to the expanded configuration. Stent 104 may be configured such that outer surface 143 radially displaces inward in a direction toward longitudinal axis L when stent 104 transitions from the expanded configuration to the deployment configuration. In some examples, stent 104 incudes a shape memory material resiliently biased to cause skeleton 112 to expand in a direction radially away from longitudinal axis L. In some examples, medical system 100 includes a balloon configured to cause skeleton 112 to expand in a direction radially away from longitudinal axis L when the balloon inflates (e.g., is caused to inflate by a clinician).

In examples, medical system 100 includes a delivery system 107 configured to cause stent 104 to transition from the deployment configuration to the expanded configuration and/or transition from the expanded configuration to the deployment configuration. Delivery system 107 be configured such that a clinician may cause stent 104 to transition from the deployment configuration to the expanded configuration and/or transition from the expanded configuration to the deployment configuration using the delivery system 107. In examples, stent 104 (e.g., at least portion of outer surface 143) is configured to contact vessel wall 144 when stent 104 is positioned within vessel 142 and in the expanded configuration.

Stent 104 defines a distal portion 146 (“stent distal portion 146”) and a proximal portion 148 (“stent proximal portion 148”) substantially opposite stent distal portion 146. Stent distal portion 146 defines a distal end 150 (“stent distal end 150”) and defines a distal opening 152 (“stent distal opening 152”). In examples, stent distal opening 152 is defined by stent distal end 150. Stent proximal portion 148 defines a proximal end 154 (“stent proximal end 154”) and defines a proximal opening 156 (“stent proximal opening 156”). In examples, stent proximal opening 156 is defined by stent proximal end 154. Stent lumen 105 extends between stent proximal portion 148 and stent distal portion 146. In examples, stent proximal opening 156 opens into stent lumen 105. Stent distal opening 152 may open into stent lumen 105. Stent 105 defines a longitudinal axis L extending through stent lumen 105. In examples, longitudinal axis L extends through stent proximal opening 156 and/or stent distal opening 152.

Stent 104 may be configured such that a blood flow of patient 102 flows through stent lumen 105 when stent 104 positioned within vessel 142. In examples, stent 104 is configured such that the blood flow flows between stent proximal opening 156 and stent distal opening 152 (e.g., via stent lumen 105). For example, stent 104 may be configured to position (e.g., to be positioned by a clinician) within vessel 142 such that a blood flow B1 flows from stent proximal opening 156 to stent distal opening 152. Stent 104 may be configured to position (e.g., to be positioned by a clinician) within vessel 142 such that a blood flow B2 flows from stent distal opening 152 to stent proximal opening 156.

In examples, skeleton 112 surrounds longitudinal axis L. Note that, although skeleton 112 is depicted as a distal portion 158 (“skeleton distal portion 158”) and a separated proximal portion 160 (“skeleton proximal portion 160”), this is mainly to provide clarity for the depiction of components of light emitter device 118 and/or other features of stent 104 which skeleton 112 may substantially surround. In examples, skeleton distal portion 158 and skeleton proximal portion 160 form a unified skeleton structure extending from stent distal portion 146 (e.g., from stent distal opening 152) to stent proximal portion 148 (e.g., to stent proximal opening 156). However, in some examples, skeleton 112 may be configured such that skeleton distal portion 158 defines a first portion of skeleton 112 and a skeleton proximal portion 160 defines a second portion of skeleton 112 which is separated from the first portion. In examples, skeleton distal portion 158 comprises some portion of stent distal portion 146. Skeleton proximal portion 160 may comprise some portion of stent proximal portion 148.

Skeleton 112 may be configured to radially expand outward in a direction away from longitudinal axis L when stent 104 transitions from the deployment configuration to the expanded configuration. In examples, skeleton 112 is configured to cause stent 104 to transition from defining the first cross-sectional dimension D1 to defining the second cross-sectional dimension D2 when skeleton 112 radially expands outward in the direction away from longitudinal axis L. In some examples, skeleton 112 is configured to radially contract inward in a direction toward longitudinal axis L when stent 104 transitions from the expanded configuration to the deployment configuration. In examples, skeleton 112 may be configured to cause stent 104 to transition from defining the second cross-sectional dimension D2 to defining the first cross-sectional dimension D1 when skeleton 112 radially contracts inward in the direction toward longitudinal axis L.

Skeleton 112 may include a plurality of interconnecting struts 162 (“struts 162”) including struts such as strut 164 and strut 166. One or more of struts 162 (e.g., strut 164, 166) may be positioned to extend over a longitudinal length of stent 104. In some examples, strut 164, 166 may extend at least partially around longitudinal axis L. In some examples, strut 164, 166 may define at least some portion of outer surface 143. Substantially adjacent struts may be connected via one or more connectors, such as connector 168. In examples, struts 162 may define apices (e.g., substantially repeating geometric units). In examples, skeleton 112 may be configured such that a first strut (e.g., strut 164) moves relative to a second strut (e.g., strut 166) when stent 104 transitions between the deployment configuration and the expanded configuration. For example, skeleton 112 may be configured such that a displacement DS between strut 164 and strut 166 increases or decreases when stent 104 transitions between the deployment configuration and the expanded configuration. In examples, skeleton 112 is configured to cause displacement DS to increase when stent 104 transitions from the deployment configuration to the expanded configuration. Skeleton 112 may be configured to cause displacement DS to decrease when stent 104 transitions from the expanded configuration to the deployment configuration.

Skeleton 112 supports one or more electrodes such as electrode 170 and/or electrode 172. Electrode 170, 172 is configured to receive (e.g., detect and/or sense) an electrical signal indicative of an EEG of patient 102. In some examples, electrode 170, 172 comprises one or more conductive fibers, such as, for example, one or more conductive fibers including gold or silver. In examples, the one or more conductive fibers extend over a longitudinal length of stent 104. In some examples, the one or more conductive fibers extend at least partially around longitudinal axis L.

In some examples, the electrode 170, 172 comprises a conductive fabric 174 supported by skeleton 112 (e.g., when conductive fibers comprise electrode 170, 172). Stent 104 may be configured such that conductive fabric 174 substantially surrounds at least some portion of longitudinal axis L. In examples, conductive fabric 174 extends from stent distal portion 146 (e.g., from stent distal opening 152) to stent proximal portion 148 (e.g., to stent proximal opening 156). In some examples, stent 104 is configured such that conductive fabric 174 substantially defines at least some portion of a boundary 176 defining stent lumen 105 (“Stent lumen boundary 176”). In examples, stent lumen boundary 176 substantially surrounds longitudinal axis L. conductive fabric 174 defines at least some portion of outer surface 143. In some examples, stent 104 is configured such that strut 164, 166 is substantially interposed between conductive fabric 174 and longitudinal axis L. In some examples, stent 104 is configured such that conductive fabric 174 is substantially interposed between strut 164, 166 and longitudinal axis L.

Electrode 170, 172 may be configured to detect an electrical signal indicative of the spontaneous bioelectric activity of the brain of patient 102. Stent 104 may be configured to communicate the electrical signal from electrode 170, 172 to processing circuitry 110. Electrode 170, 172 may be configured to detect electrical signals which include features representative of the bioelectric activity, such as amplitudes and/or frequencies in one or more frequency bands. In examples, electrode 170, 172 is configured to detect electrical signals in the frequency bands of at least a delta band (e.g., 0.5-4 Hz) and an alpha band (e.g., from about 8-12 Hz, including an alpha1 band of about 8-10 Hz and an alpha2 band of about 10-12 Hz). Electrode 170, 172 may be configured to detect electrical signals in additional frequency bands, such as theta (e.g., 4-7 Hz), beta (e.g. 13-30 Hz) and gamma (e.g., 30-40 Hz) bands. In examples, electrode 170, 172 is configured to detect electrical signals in a high gamma band (e.g., for use in seizure monitoring and detection), such as frequencies greater than about 40 Hz in some examples, frequencies within a band of about 30-60 Hz in some examples, and/or frequencies of about 30-100 Hz in some examples. In examples, electrode 170, 172 is configured to detect an electrical signal having an amplitude of less than about 100 microvolts (μV) when compared to a reference voltage (e.g., a reference voltage provided by electrode 111 (FIG. 1)). In some examples, electrode 170, 172 is configured to detect an electrical signal having an amplitude of less than about 30 microvolts (μV) when compared to the reference voltage. Processing circuitry 110 may include brain signal analysis circuitry, which may be configured to the features representative of the bioelectric activity (e.g., amplitudes and/or frequencies in one or more of the delta band, alpha band, theta band, beta band, and gamma band) using the electrical signal sensed by electrode 170, 172.

Stent 104 may be configured to determine an oxygen level of blood within stent lumen 105 and/or otherwise surrounding stent 104 using light emitter device 118. In examples, stent 104 (e.g., skeleton 112) supports light emitter device 118. Stent 104 may support light emitter device 118 such that the light emitted is projected at least within stent lumen 105. In examples, light emitter device 118 includes a housing 178 (“light device housing 178”) configured to support lens 124 and/or waveguide 120. Stent 104 (e.g., skeleton 112) may be configured to support light device housing 178. In examples, light device housing 178 is configured to position lens 124 and/or waveguide 120 such that light emitter device 118 emits light (e.g., light generated by light source 122) into stent lumen 105 when stent 104 supports light device housing 178. Light emitter device 118 is configured to project the light to cause an acoustic response of blood cells (e.g., hemoglobin) within stent lumen 105. The acoustic response may be expected to cause an acoustic wave (e.g., a pressure wave) to propagate through the blood within stent lumen 105.

Stent 104 may include a first material 180 configured to detect a first acoustic wave portion. In examples, first material 180 includes a first elongate fiber 182. First elongate fiber 182 may be a conductive fiber. In some examples, first material 180 (e.g., first elongate fiber 182) is substantially supported by skeleton 112 and/or conductive fabric 174. For example, first elongate fiber 182 may be substantially woven into conductive fabric 174. In examples, stent 104 is configured such that first material 180 substantially surrounds at least some portion of longitudinal axis L. extending through the stent lumen. Stent 104 is configured to provide (e.g., via comm link 115) a first signal indicative of the first acoustic wave portion detected by first material 180.

Stent 104 may include a second material 184 configured to detect a second acoustic wave portion. In examples, second material 184 includes a second material portion 186 supported by light emitter device 118 (e.g., supported in substantial proximity to lens 124 and/or waveguides 120). In some examples, second material 184 comprises a second elongate fiber 188 substantially supported by skeleton 112 and/or conductive fabric 174. In some examples, stent 104 is configured such that second material 184 substantially surrounds at least some portion of longitudinal axis L. Stent 104 is configured to provide (e.g., via comm link 117 or a comm link 119) a second signal indicative of the second acoustic wave portion detected by second material 180.

Processing circuitry 110 is configured to determine an EEG parameter using the EEG signal provided by stent 104. Processing circuitry 110 may be configured to determine an oxygen parameter using the oxygen signal received from stent 104, and may be configured to determine a flow parameter using the flow signal received from stent 104. Processing circuitry 110 may be configured to compare the EEG parameter to an EEG threshold, compare the oxygen parameter to an oxygen threshold, and/or compare the flow parameter to a flow threshold. Processing circuitry 110 may be configured to indicate that patient 102 may have experienced a stroke based on one or more of the comparison of the EEG parameter and the EEG threshold, the comparison of the oxygen parameter and the oxygen threshold, and the comparison of the flow parameter and the flow threshold.

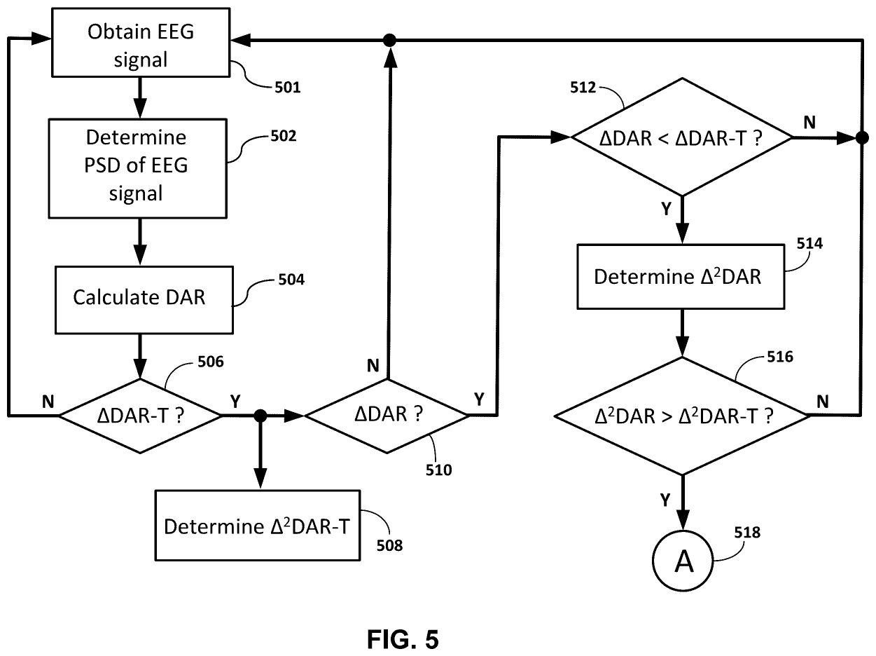

For example, FIG. 5 depicts an example technique by which processing circuitry 110 may compare the EEG parameter to the EEG threshold. Processing circuitry 110 may be configured to compare the EEG parameter to the EEG threshold using other techniques in other examples.

Processing circuitry 110 may obtain the EEG signal from stent 104 (e.g., using comm link 114) (501). The EEG signal may be indicative of the electrical signal sensed by electrode 170, 172 (e.g., conductive fabric 174). In examples, the EEG signal is indicative of the spontaneous bioelectric activity of the brain of patient 102. In examples, processing circuitry 110 obtain the EEG signal at least when stent 104 is positioned within anatomical volume 106 (e.g., vessel 142) and stent 104 is in the expanded configuration. In examples, processing circuitry 110 obtains the EEG signal over a range of at least approximately 0.5 Hertz (Hz) to at least approximately 200 Hz. The EEG signal may comprise a waveform which may be represented as a composite waveform comprised of a plurality of waveforms having different frequencies. For example, the EEG signal may comprise a waveform which may be represented as a composite of at least a delta waveform having a frequency between about 0.5 Hz and about 4 Hz and an alpha waveform having a frequency of about 8 Hz and about 13 Hz. In examples, the EEG signal may be represented as composite waveform comprising at least the delta waveform, the alpha waveform, a theta waveform having a frequency between about 4 Hz and about 8 Hz, a beta waveform having a frequency between about 13 Hz and about 30 Hz, and a gamma waveform having a frequency between about 30 Hz and about 200 Hz.

Processing circuitry 110 determines a spectral power density (PSD) using the EEG signal (502). The PSD may be indicative of a power distribution of the EEG signal in a frequency domain (e.g., a frequency domain including frequencies of delta waveform, the alpha waveform, the theta waveform, the beta waveform, and/or the gamma waveform). In examples, processing circuitry 110 determines the PSD using a segmentation method capable of distributing a power of the EEG signal over a frequency domain, such as the Welch Method, the Burg Method, or another method.

Processing circuitry 110 determines the EEG threshold by receiving the EEG signal over an initial period when patient 102 is assessed (e.g., by a clinician) to be medically stable. For example, in some examples, the initial period may be period of time following a stroke suffered by patient 102, but during which patient 102 is assessed to be medically stable. In examples, the EEG threshold is based on one or more PSDs determined during the initial period. In some examples, the EEG threshold is based on one or more of a delta wave power indicated by a PSD and/or an alpha wave power indicated by the PSD. For example, the EEG threshold may be based on a delta-to-alpha ratio (DAR) indicative of a ratio of the delta wave power to the alpha wave power of a given PSD. In examples, processing circuitry calculates a DAR using the EEG signal (504).

In some examples, the EEG threshold is based on a DAR threshold difference determined during the initial period. For example, processing circuitry 110 may determine a primary DAR-T1 using a primary PSD determined over a primary time period, where the primary time period is a chronological period within the initial period. The primary DAR-T1 may be indicative of a ratio of a primary delta wave power of the primary PSD to a primary alpha wave power of the primary PSD. Processing circuitry 110 may determine a secondary DAR-T2 using a second PSD determined over a secondary time period, where the secondary time period is another chronological period within the initial period. In examples the secondary time period is subsequent to the primary time period. The secondary DAR-T2 may be indicative of a ratio of a secondary delta wave power of the secondary PSD to a secondary alpha wave power of the secondary PSD. Processing circuitry 110 may determine the EEG threshold based on a difference between the primary DAR-T1 and the secondary DAR-T1.

For example, processing circuitry 110 may determine the primary DAR-T1 over the primary time period using 501, 502, and 504 of FIG. 5. Processing circuitry 110 may determine if sufficient DARs over the initial period have been determined to allow for determination of a DAR threshold difference (e.g., if there is at least a primary DAR-T1 and a secondary DAR-T2). If not (e.g., “N” at 506), processing circuitry 110 return to 501, 502, and 504 over the secondary time period to determine the secondary DAR-T2. When processing circuitry 110 has determined the primary DAR-T1 and the secondary DAR-T2 (“Y” at 506), processing circuitry 110 may determine the EEG threshold (“ΔDAR-T”) based on a difference between the primary DAR-T1 and the secondary DAR-T1. Hence, in examples, the EEG threshold may be indicative of change in a DAR of patient 102 that might be observed and/or expected when patient 102 is assessed to be (e.g., by a clinician) in a medically stable state.

As discussed below, processing circuitry 110 is configured to substantially monitor patient 102 by receiving an EEG parameter and comparing the EEG parameter to the EEG threshold. Processing circuitry 110 may be configured to indicate that patient 102 may have experienced a stroke event based on the comparison.

Processing circuitry 110 may subsequently continue to receive the EEG signal and monitor changes to the determined DAR to monitor the condition of patient 102. Processing circuitry 110 may generate an EEG alert based on a comparison of an EEG parameter indicative of a received EEG signal and the EEG threshold. Hence, processing circuitry 110 may provide an indication that patient 102 may have suffered a stroke (e.g., a subsequent stroke) subsequent to determination of the EEG threshold.

For example, processing circuitry 110 may determine an EEG parameter by receiving the EEG signal over a monitoring period subsequent to the initial period. In examples, the EEG parameter is based on one or more PSDs determined during the monitoring period. In some examples, the EEG parameter is based on one or more of a delta wave power indicated by a PSD during the monitoring period and/or an alpha wave power indicated by the PSD during the monitoring period. For example, the EEG threshold may be based on a delta-to-alpha ratio (DAR) indicative of a ratio of the delta wave power to the alpha wave power of the PSD during the monitoring period.

In some examples, processing circuitry 110 determines a time rate of change of the ΔDAR-T (e.g., “Δ²DAR-T”) (508). In examples, the time rate of change is a second order differential of ΔDAR-T. For example, processing circuitry 110 may determine the time rate of change based on a difference between the primary DAR-T1 and the secondary DAR-T2 and a chronological difference between the primary time period and the secondary time period. The difference between the primary time period and the secondary time period may be, for example, indicative of a difference between a given time mark encompassed by the primary time period and a given time mark encompassed by the secondary time period. Although represented as occurring substantially immediately after determining the EEG threshold (“ΔDAR-T”) on FIG. 5, this is not required. Processing circuitry 110 may determine the time rate of change of the ΔDAR-T at other points of FIG. 5 in other examples.

Processing circuitry 110 may be configured to determine the EEG parameter by continuing to receive the EEG signal subsequent to the determination of the EEG threshold (“ΔDAR-T”). For example, when ΔDAR-T has been determined, such that processing circuitry 110 follows “Y” at 506, processing circuitry 110 may return to 501 (e.g., via 510) to continue to receive the EEG signal. Processing circuitry 110 may proceed through one or more of 501, 502, 504, and/or 506 to determine the EEG parameter. In some examples, the EEG parameter is based on a DAR difference (“ΔDAR”) determined during the monitoring period.

For example, processing circuitry 110 may determine a first DAR-1 using a first PSD determined over a first time period, where the first time period is a chronological period within the monitoring period. The first DAR-1 may be indicative of a ratio of a first delta wave power of the first PSD to a first alpha wave power of the first PSD. Processing circuitry 110 may determine a second DAR-2 using a second PSD determined over a second time period, where the second time period is another chronological period within the monitoring period. In examples the second time period is subsequent to the first time period. The second DAR-2 may be indicative of a ratio of a second delta wave power of the second PSD to a second alpha wave power of the second PSD. Processing circuitry 110 may determine the EEG parameter based on a difference between the first DAR-1 and the second DAR-1.

As an example, processing circuitry 110 may determine the first DAR over the first time period using 501, 502, 504, and/or 506 of FIG. 5 (e.g., when processing circuitry 110 follows “Y” at 506). Processing circuitry 110 may determine if sufficient DARs over the first time have been determined to allow for determination of a DAR difference (e.g., if there is at least a first DAR-1 and a second DAR-2). If not (e.g., “N” at 510), processing circuitry 110 may again conduct 501, 502, 504, and/or 506 over the second time period to determine the second DAR-2. When processing circuitry 110 has determined the first DAR-1 and the second DAR-2 (“Y” at 510), processing circuitry 110 may determine the EEG parameter (e.g. “ΔDAR”) based on a difference between the first DAR-1 and the second DAR-2. Hence, in examples, the EEG parameter may be indicative of change in a DAR of patient 102 observed during the monitoring period.

Processing circuitry 110 may compare the EEG parameter (e.g., “ΔDAR”) and the EEG threshold (e.g., “ΔDAR-T”) to assess a condition of patient 102 during the monitoring period relative to the condition of patient 102 during the initial period (512). For example, when the EEG parameter (e.g., “ΔDAR”) has been determined and processing circuitry 110 follows “Y” at 510, processing circuitry 110 may compare the EEG parameter and the EEG threshold at 512. Processing circuitry 110 may generate the EEG alert based on the comparison of the EEG parameter and the EEG threshold. For example, if the comparison of the EEG parameter and the EEG threshold sufficient for the EEG alert, processing circuitry 110 may proceed toward issuing the EEG alert (e.g., via the “Y” path of 512). If the comparison of the EEG parameter and the EEG threshold does not satisfy a condition sufficient for the EEG alert, processing circuitry 110 may continue to receive the EEG signal (e.g., via the “N” path of 512), determine a subsequent EEG parameter (e.g., via 501, 502, 504, 506, and/or 510), and compare the subsequent EEG parameter to the EEG threshold. Processing circuitry 110 may continue to determine subsequent EEG parameters on a substantially ongoing basis to continue to monitor the condition of patient 102.

In some examples, processing circuitry 110 compares the EEG parameter and the EEG threshold (e.g., at 512) and proceeds toward issuing the EEG alert (e.g., via the “Y” path of 512) when the EEG parameter is less than the EEG threshold. For example, processing circuitry 110 may proceed toward issuing the alert when ΔDAR is less than ΔDAR-T. In some examples, (e.g., when ΔDAR is less than ΔDAR-T), processing circuitry 110 determines a time rate of change of the ΔDAR (e.g., “Δ²DAR”) (514).

In examples, the time rate of change of the ΔDAR is a second order differential of ΔDAR. For example, processing circuitry 110 may determine the time rate of change based on a difference between the first DAR and the second DAR and a chronological difference between the first time period and the second time period. The difference between the first time period and the second time period may be, for example, indicative of a difference between a given time mark encompassed by the first time period and a given time mark encompassed by the second time period.

Processing circuitry 110 may compare the time rate of change of the ΔDAR (e.g., Δ²DAR) and the time rate of change of the ΔDAR-T (e.g., Δ²DAR-T) and issue the EEG alert based on the comparison (516). For example, processing circuitry 110 may issue the EEG alert (“A”) at 518. For example, if the comparison of the Δ²DAR and the Δ²DAR-T satisfies a criteria sufficient for the EEG alert, processing circuitry 110 may proceed toward issuing the EEG alert (e.g., via the “Y” path of 516). In examples, processing circuitry 110 proceeds towards issuing the EEG alert if the time rate of change of the ΔDAR is greater than the time rate of change of the ΔDAR-T (e.g., if Δ²DAR is greater than Δ²DAR-T). If the comparison of the Δ²DAR and the Δ²DAR-T does not satisfy a condition sufficient for the EEG alert, processing circuitry 110 may continue to receive the EEG signal (e.g., via the “N” path of 516), determine a subsequent ΔDAR (e.g., via 501, 502, 504, 506, and/or 510) and compare the ΔDAR and the ΔDAR-T (e.g., at 512). Processing circuitry may compare the time rate of change of the subsequent ΔDAR to the time rate of change of the ΔDAR-T based on the relationship between the ΔDAR and the ΔDAR-T. Processing circuitry 110 may continue to determine subsequent ΔDARs on a substantially ongoing basis to continue to monitor the condition of patient 102.

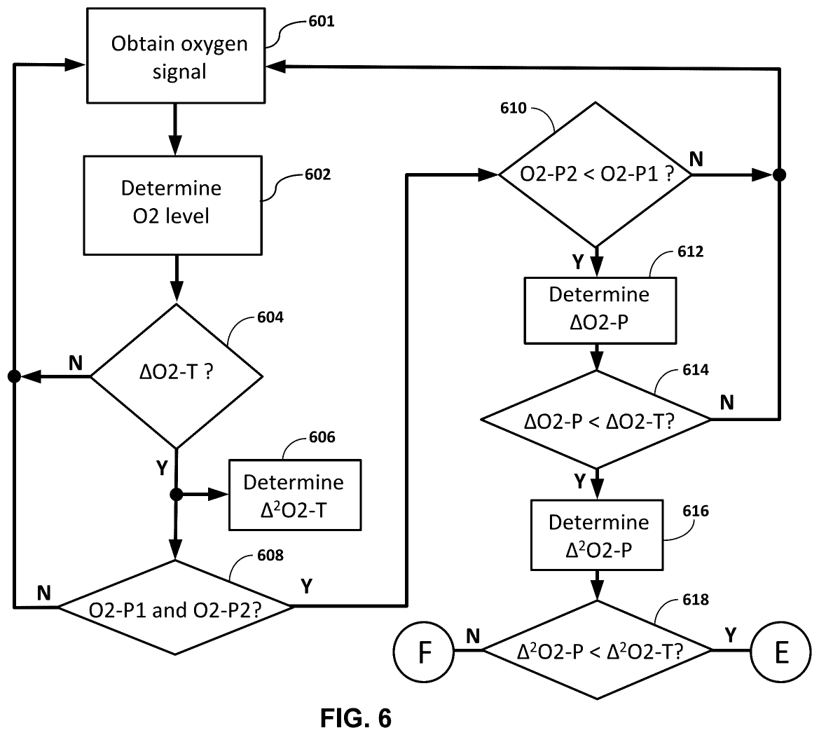

FIG. 6 depicts an example technique by which processing circuitry 110 may compare the oxygen parameter to the oxygen threshold. Processing circuitry 110 may be configured to compare the oxygen parameter to the oxygen threshold using other techniques in other examples.

Processing circuitry 110 may obtain the oxygen signal from stent 104 using comm link 115 and/or comm link 117) (601). In some examples, the oxygen signal is indicative of the first electrical parameter and/or alteration thereof produced by first material 180 in response to interaction with the first acoustic wave portion caused by light emitted by light emitter device 118. In some examples, the oxygen signal is indicative of the second electrical parameter and/or alteration thereof produced by second material 184 in response to interaction with the second acoustic wave portion caused by light emitted by light emitter device 118. In examples, the oxygen signal is indicative of an oxygen level of blood within the anatomical volume 106. In examples, the oxygen signal is indicative of an SjVO2 of patient 102 (e.g., when vessel 142 is a jugular vein). Processing circuitry 110 may obtain the oxygen signal at least when stent 104 is positioned within anatomical volume 106 (e.g., vessel 142) and stent 104 is in the expanded configuration. Processing circuitry 110 is configured to determine an oxygen level (“O2 level”) using the oxygen signal (602).

Processing circuitry 110 is configured to use the oxygen level to compare an oxygen parameter to an oxygen threshold. In examples, processing circuitry 110 substantially determines a trend of the oxygen level. In some examples, processing circuitry 110 issues a first oxygen alert, a second oxygen alert, or a third oxygen alert based on the comparison of the oxygen parameter and the oxygen threshold. As will be discussed, processing circuitry 110 may treat the first oxygen alert as indicative that patient 102 may be experiencing or have experienced a hemorrhagic stroke. Processing circuitry 110 may treat the second oxygen alert and/or the third oxygen alert as indicative that patient 102 may be experiencing or have experienced an ischemic stroke.

Processing circuitry 110 determines the oxygen threshold by receiving the oxygen signal over the initial period when patient 102 is assessed (e.g., by a clinician) to be medically stable. In some examples, the oxygen threshold is based on an O2 threshold difference determined during the initial period, where the O2 threshold difference is based on a comparison of a first O2 threshold level and a second O2 threshold level. For example, processing circuitry 110 may determine the first O2 threshold level using an initial oxygen signal received from stent 104 during the initial period. Processing circuitry 110 may determine the second O2 threshold level using a subsequent oxygen signal received from stent 104 during the initial period. Processing circuitry 110 may determine the oxygen threshold based on a difference between the first O2 threshold level and the second O2 threshold level. In examples, processing circuitry 110 determines the oxygen threshold only if the second O2 threshold level is less than the first oxygen threshold level.

For example, processing circuitry 110 may determine the first O2 threshold level using 601 and 602 of FIG. 6. Processing circuitry 110 may determine if sufficient oxygen levels have been determined to allow for determination of the oxygen threshold difference (e.g., if there is at least a first O2 threshold level and a second O2 threshold level). If not (e.g., “N” at 604), processing circuitry 110 return to 601 and 602 to determine the second O2 threshold level. When processing circuitry 110 has determined at least the first O2 threshold level and the second O2 threshold level, processing circuitry 110 may determine the oxygen threshold (“ΔO2-T”) based on a difference between the first O2 threshold level and the second O2 threshold level.

In examples, the oxygen threshold is indicative of a decreasing trend of an oxygen level of patient 102. For example, in some examples, processing circuitry 110 determines the oxygen threshold (e.g., ΔO2-T) based on the difference between the first O2 threshold level and the second O2 threshold level only if the second O2 threshold level is less than the first oxygen threshold level. For example, at 604, if the second O2 threshold level is not less than the first O2 threshold level, processing circuitry 110 may return to 601 (e.g., via “N” of 604). Processing circuitry 110 may obtain a subsequent oxygen threshold level using 601 and 602. Processing circuitry 110 may continue to obtain subsequent oxygen threshold levels until a subsequent oxygen threshold level indicates the oxygen level of patient 102 may be decreasing (e.g., when the subsequent oxygen level is compared to the first threshold oxygen level, the second threshold oxygen level, or another of the subsequent oxygen thresholds). Hence, in examples, the oxygen threshold (e.g., ΔO2-T) may be indicative of a decrease in an oxygen level of patient 102 that could be expected when patient 102 is assessed to be (e.g., by a clinician) in a medically stable state.

In some examples, processing circuitry 110 determines a time rate of change of the oxygen threshold (e.g., “Δ²O2-T”) (606). In examples, the time rate of change is a second order differential of ΔO2-T. For example, processing circuitry 110 may determine the time rate of change based on a difference between the first oxygen threshold level and the second oxygen threshold level and a chronological difference between a first time mark associated with the first oxygen threshold level and a second time mark associated with the second oxygen threshold level. Although represented as occurring substantially immediately after determining the oxygen threshold (“ΔO2-T”) on FIG. 6, this is not required. Processing circuitry 110 may determine the time rate of change of the oxygen threshold at other points of FIG. 6 in other examples.

As discussed below, processing circuitry 110 is configured to substantially monitor patient 102 by receiving an oxygen parameter and comparing the oxygen parameter to the oxygen threshold. Processing circuitry 110 may be configured to indicate that patient 102 may have experienced a stroke event based on the comparison.

Processing circuitry 110 may be configured to determine the oxygen parameter by continuing to receive the oxygen signal subsequent to the determination of the oxygen threshold (“ΔO2-T”). For example, when ΔO2-T has been determined, such that processing circuitry 110 follows “Y” at 604, processing circuitry 110 may return to 601 (e.g., via 610) to continue to receive the oxygen signal. Processing circuitry 110 may proceed through one or more of 601, 602, and/or 604 to determine the oxygen parameter. In some examples, the oxygen parameter is based on an oxygen parameter difference (“ΔO2-P”) determined during the monitoring period.

In some examples, the oxygen parameter is based on an O2 parameter difference determined during the monitoring period, where the O2 parameter difference is based on a comparison of a first O2 parameter level (“O2-P1”) and a second O2 parameter level (“O2-P2”). For example, processing circuitry 110 may determine O2-P1 using a first oxygen signal received from stent 104 during the monitoring period. Processing circuitry 110 may determine O2-P2 using a second oxygen signal received from stent 104 during the monitoring period. Processing circuitry 110 may determine the oxygen parameter based on comparison of O2-P1 and O2-P1. In examples, processing circuitry 110 determines the oxygen parameter based on a difference between the O2-P1 and O2-P2.

For example, processing circuitry 110 may determine the O2-P1 using 601 and 602 of FIG. 6. Processing circuitry 110 may determine if sufficient oxygen levels have been determined to allow for determination of the O2 parameter difference (e.g., if there is at least a O2-P1 and an O2-P2). If not (e.g., “N” at 608), processing circuitry 110 return to 601 and 602 to determine O2-P2. When processing circuitry 110 has determined O2-P1 and O2-P2, processing circuitry 110 may determine the oxygen parameter (“ΔO2-P”) based on a comparison (e.g., a difference between) O2-P1 and O2-P2. Processing circuitry 110 may determine the oxygen parameter (e.g., ΔO2-P) at 612.

In examples, the oxygen parameter is indicative of a decreasing trend of an oxygen level of patient 102. For example, in some examples, processing circuitry 110 determines the oxygen parameter (e.g., ΔO2-P) by comparing O2-P1 and the O2-P2 only if O2-P2 is less than O2-P1. For example, at 610, if O2-P2 is not less than O2-P1, processing circuitry 110 may return to 601 (e.g., via “N” of 610). Processing circuitry 110 may obtain a subsequent oxygen parameter level using 601, 602, 604, and/or 608. Processing circuitry 110 may continue to obtain subsequent oxygen parameters levels (e.g., via “N” of 610) until a subsequent oxygen parameter level indicates the oxygen level of patient 102 may be decreasing (e.g., when the subsequent oxygen level is compared to O2-P1, O2-P2, or another of the subsequent oxygen parameter levels).

Processing circuitry 110 may compare the oxygen parameter (e.g., ΔO2-P) and the oxygen threshold (e.g., ΔO2-T) to assess a condition of patient 102 during the monitoring period relative to the condition of patient 102 during the initial period (614). Processing circuitry 110 may generate the oxygen alert based on the comparison of the oxygen parameter and the oxygen threshold. For example, if the comparison of the oxygen parameter and the oxygen threshold satisfies a criteria sufficient for the oxygen alert, processing circuitry 110 may proceed toward issuing the oxygen alert (e.g., via the “Y” path of 614). If the comparison of the oxygen parameter and the oxygen threshold does not satisfy a condition sufficient for the oxygen alert, processing circuitry 110 may continue to receive the oxygen signal (e.g., via the “N” path of 614), determine subsequent oxygen parameters (e.g., via 601, 602, 604, 608, and/or 612), and compare the subsequent oxygen parameter to the oxygen threshold. Processing circuitry 110 may continue to determine subsequent oxygen parameters on a substantially ongoing basis to continue to monitor the condition of patient 102.

In some examples, processing circuitry 110 compares the oxygen parameter and the oxygen threshold (e.g., at 610) and proceeds toward issuing the oxygen alert (e.g., via the “Y” path of 614) when the oxygen parameter is less than the oxygen threshold. For example, processing circuitry 110 may proceed toward issuing the alert when ΔO2-P is less than ΔO2-T.

In some examples, processing circuitry 110 determines a time rate of change of the oxygen parameter (e.g., “Δ²O2-P”) (616). In examples, the time rate of change is a second order differential of ΔO2-P. For example, processing circuitry 110 may determine the time rate of change based on a difference between the first oxygen parameter level (O2-P1) and the second oxygen parameter level (O2-P2) and a chronological difference between a first time mark associated with the first oxygen threshold level and a second time mark associated with the second oxygen threshold level.

Processing circuitry 110 may compare the time rate of change of the oxygen parameter (e.g., Δ²O2-P) and the time rate of change of the oxygen threshold (e.g., Δ²O2-T) and issue the oxygen alert based on the comparison (618). The oxygen alert may be at least one of a first oxygen alert or a second oxygen alert. In examples, processing circuitry 110 issues the first oxygen alert (“F”) if the time rate of change of the oxygen parameter is greater than the time rate of change of the oxygen threshold (e.g., if Δ²O2-P is greater than Δ²O2-T). In some examples, processing circuitry 110 issues the second oxygen alert (“E”) if the time rate of change of the oxygen parameter is less than the time rate of change of the oxygen threshold (e.g., if Δ²O2-P is less than Δ²O2-T).

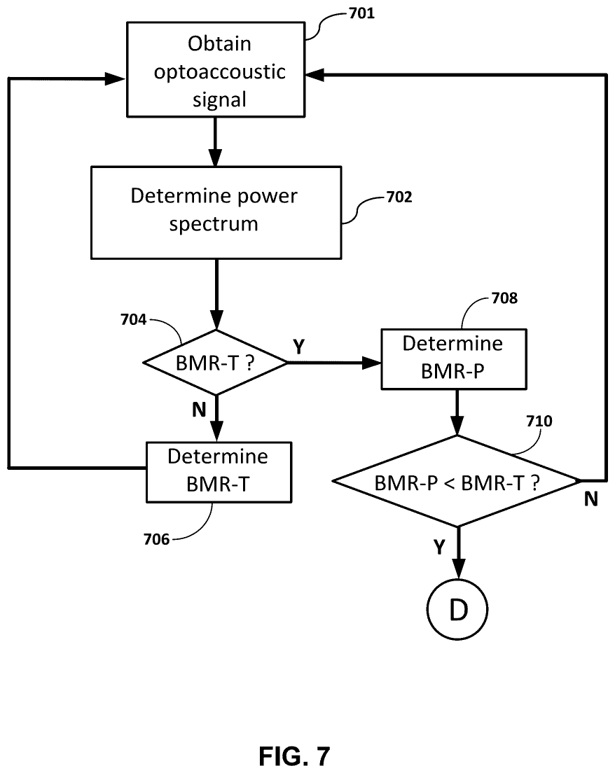

In some examples, processing circuitry 110 may compare the oxygen parameter to the oxygen threshold using photoacoustic spectral signals. FIG. 7 depicts an example technique by which processing circuitry 110 may compare the oxygen parameter to the oxygen threshold using photoacoustic spectral signals. Processing circuitry 110 may be configured to compare the oxygen parameter to the oxygen threshold using other techniques in other examples.

Processing circuitry 110 may obtain the oxygen signal from stent 104 using comm link 115 and/or comm link 117) (701). Stent 104 may provide the oxygen signal using at least one of first material 180 and/or second material 184. In examples, processing circuitry 110 obtains the oxygen signal over a frequency range including at least a B wave having a frequency between about 0.01 and 0.05 Hz (e.g., about 0.035 Hz) and an M wave having a frequency between about 0.05 Hz to about 0.15 Hz (e.g., about 0.1 Hz). The oxygen signal may comprise a waveform which may be represented as a composite waveform comprised of a plurality of waveforms having different frequencies. For example, the oxygen signal may comprise a waveform which may be represented as a composite of at least a B waveform and an M waveform.

Processing circuitry 110 determines an oxygen spectral power density (“oxygen PSD”) using the oxygen signal (702). The oxygen PSD may be indicative of a power distribution of the oxygen signal in a frequency domain (e.g., a frequency domain including frequencies of B waveform and/or the M waveform). In examples, processing circuitry 110 determines the oxygen PSD using a segmentation method capable of distributing a power of the oxygen signal over a frequency domain, such as the Welch Method, the Burg Method, or another method.

Processing circuitry 110 determines the oxygen threshold by receiving the oxygen signal over the initial period when patient 102 is assessed (e.g., by a clinician) to be medically stable. In examples, the oxygen threshold is based on one or more oxygen PSDs determined during the initial period. In some examples, the oxygen threshold is based on one or more of a B wave power indicated by an oxygen PSD and/or an M wave power indicated by the oxygen PSD. For example, the oxygen threshold may be based on a B wave-to-M wave ratio (“BMR”) indicative of a ratio of the B wave power to the M wave power of a given oxygen PSD.

For example, processing circuitry 110 may determine the oxygen threshold using a first oxygen signal received from stent 104 during the initial period at 701. The first oxygen signal may comprise a waveform which may be represented as a first B wave having a B wave frequency (e.g., a frequency of about 0.035 Hz) and a first M wave having an M wave frequency (e.g., a frequency of about 0.1 Hz). Processing circuitry 110 may determine a first oxygen PSD using the first oxygen signal and determine a BMR of the first oxygen PSD at 702. Processing circuitry 110 may evaluate if an oxygen threshold (e.g., BMR-T) has been determined at 704 and determine a threshold BMR (“BMR-T”) at 706 using the BMR of the first oxygen PSD (e.g., via “N” at 704). The threshold BMR may be indicative of a ratio of the B wave power of the first B wave to the M wave power of the first M wave of the waveform comprising the first oxygen signal. Hence, in examples, the oxygen threshold may be indicative of a BMR of patient 102 that might be expected when patient 102 is assessed to be (e.g., by a clinician) in a medically stable state.

Processing circuitry 110 may determine an oxygen parameter by receiving the oxygen signal over the monitoring period subsequent to the initial period. In examples, the oxygen parameter is based on one or more oxygen PSDs determined during the monitoring period. For example, the oxygen parameter may be based on one or more of a B wave power indicated by an oxygen PSD determined during the monitoring period and/or an M wave power indicated by the oxygen PSD determined during the monitoring period. In examples, the oxygen parameter is based on a B wave-to-M wave ratio (“BMR”) indicative of a ratio of the B wave power of the oxygen PSD determined during the monitoring period to the M wave power of the oxygen PSD determined during the monitoring period.

Processing circuitry 110 may be configured to determine the oxygen parameter by continuing to receive the oxygen signal subsequent to the determination of the oxygen threshold (e.g., BMR-T). For example, when BMR-T has been determined, such that processing circuitry 110 follows “Y” at 704, processing circuitry 110 may return to 701 to continue to receive the oxygen signal. Processing circuitry 110 may proceed through one or more of 701, 702, and/or 704 to determine the oxygen parameter. In some examples, the oxygen parameter is based on a BMR determined during the monitoring period.

For example, processing circuitry 110 may determine the oxygen parameter using a second oxygen signal received from stent 104 during the monitoring period at 701. The second oxygen signal may comprise a waveform which may be represented as a second B wave substantially having the B wave frequency (e.g., a frequency of about 0.035 Hz) and a second M wave substantially having the M wave frequency (e.g., a frequency of about 0.1 Hz). Processing circuitry 110 may determine a second oxygen PSD using the second oxygen signal and determine a BMR of the second oxygen PSD at 702. Processing circuitry 110 may determine a parameter BMR (“BMR-P”) at 708 using the BMR of the second oxygen PSD (e.g., via “Y” at 704). The parameter BMR may be indicative of a ratio of the B wave power of the second B wave to the M wave power of the second M wave of the waveform comprising the second oxygen signal.

Processing circuitry 110 may compare the oxygen parameter (e.g., BMR-P) and the oxygen threshold (e.g., BMR-T) to assess a condition of patient 102 during the monitoring period relative to the condition of patient 102 during the initial period (710). Processing circuitry 110 may generate an oxygen alert (e.g., a third oxygen alert (“D”)) based on the comparison of the oxygen parameter and the oxygen threshold. For example, if the comparison of the oxygen parameter and the oxygen threshold satisfies a criteria sufficient for the oxygen alert, processing circuitry 110 may proceed toward issuing the oxygen alert (e.g., via the “Y” path of 710). If the comparison of the oxygen parameter and the oxygen threshold does not satisfy a condition sufficient for the oxygen alert, processing circuitry 110 may continue to receive the oxygen signal (e.g., via the “N” path of 710), determine subsequent oxygen parameters (e.g., via 701, 702, 704, and/or 708), and compare the subsequent oxygen parameter to the oxygen threshold. Processing circuitry 110 may continue to determine subsequent oxygen parameters on a substantially ongoing basis to continue to monitor the condition of patient 102.

In some examples, processing circuitry 110 compares the oxygen parameter and the oxygen threshold (e.g., at 710) and proceeds toward issuing the third oxygen alert (e.g., via the “Y” path of 710) when the oxygen parameter is less than the oxygen threshold. For example, processing circuitry 110 may proceed toward issuing the third oxygen alert when BMR-P is less than BMR-T.

Processing circuitry 110 is configured to determine a flow threshold and one or more flow parameters using the flow signal provided by stent 104. Processing circuitry 110 may obtain the flow signal from stent 104 using comm link 115 and/or comm link 117) (601). In some examples, the flow signal is indicative of the first electrical parameter and/or alteration thereof produced by first material 180 in response to interaction with the first acoustic wave portion caused by light emitted by light emitter device 118. In some examples, the flow signal is indicative of the second electrical parameter and/or alteration thereof produced by second material 184 in response to interaction with the second acoustic wave portion caused by light emitted by light emitter device 118. The flow signal may be indicative of a flow rate of blood within the anatomical volume 106. In examples, the flow signal is indicative of a cerebral blood flow of patient 102 (e.g., when vessel 142 is a jugular vein). Processing circuitry 110 may obtain the flow signal at least when stent 104 is positioned within anatomical volume 106 (e.g., vessel 142) and stent 104 is in the expanded configuration.

Processing circuitry 110 is configured to use the flow signal to compare a flow parameter to a flow threshold. In some examples, processing circuitry 110 issues a first flow alert or a second flow alert based on the comparison of the flow parameter and the flow threshold. As will be discussed, processing circuitry 110 may treat the first flow alert as indicative that patient 102 may be experiencing or have experienced a hemorrhagic stroke. Processing circuitry 110 may treat the second flow alert as indicative that patient 102 may be experiencing or have experienced an ischemic stroke.

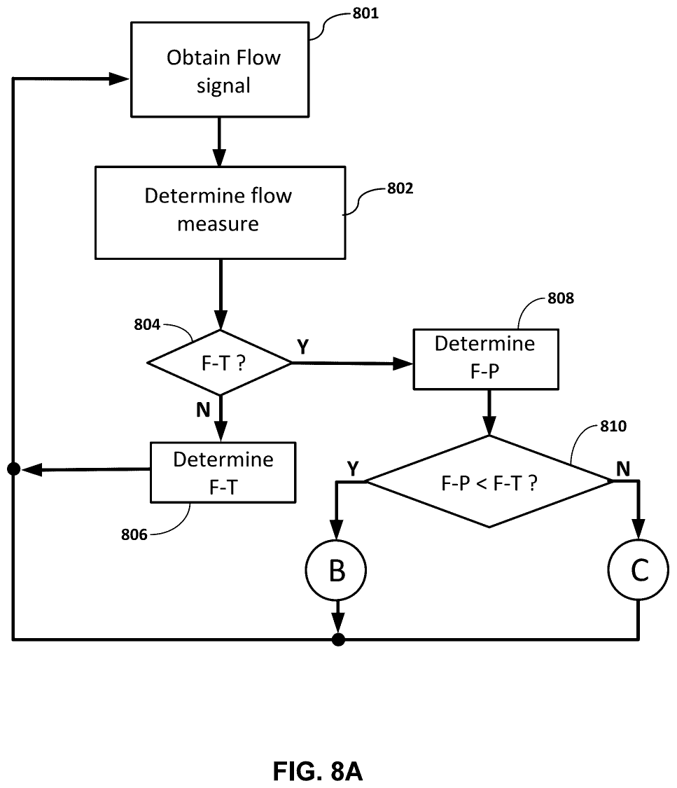

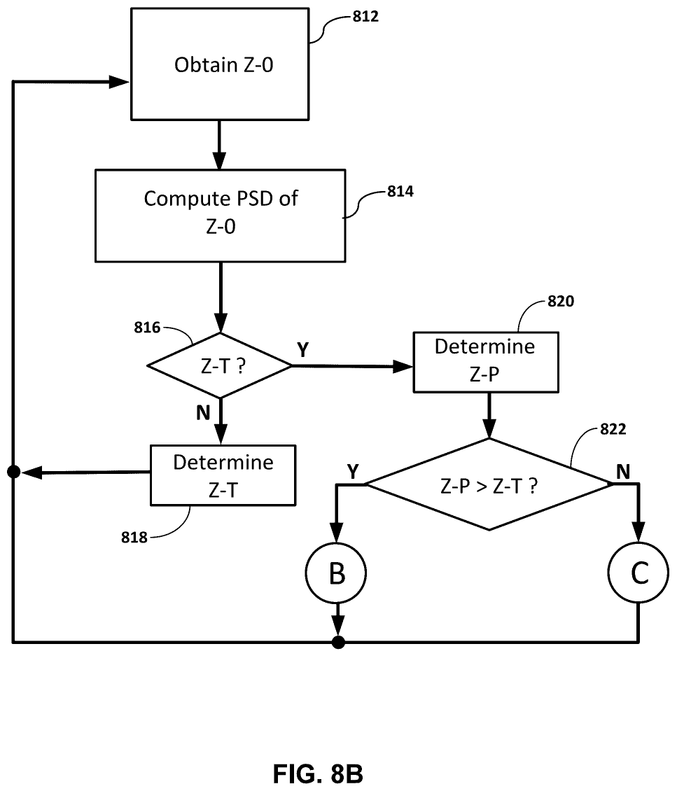

FIG. 8A depicts an example technique by which processing circuitry 110 may compare the flow parameter to the flow threshold. Processing circuitry 110 may be configured to compare the flow parameter to the flow threshold using other techniques in other examples.

Processing circuitry 110 determines the flow threshold by receiving the flow over the initial period when patient 102 is assessed (e.g., by a clinician) to be medically stable. In examples, the flow threshold is based on one or more photoacoustic images determined during the initial period, a doppler flux determined during the initial period, or another manner using a flow signal during the initial period.

For example, processing circuitry 110 may determine the flow threshold using a first flow signal received from stent 104 during the initial period at 801. The first flow signal may be caused by a first acoustic response caused by light emitter device 118 during the initial period. For examples, light emitter device 118 may illuminate blood within anatomical volume 106 with light including a first wavelength (e.g., a wavelength less than 805 nm, such as about 700 nm) and a second wavelength greater than the first wavelength (e.g. a wavelength greater than or equal to 805 nm, such as a wavelength of about 1064 nm) to cause the first acoustic response.

Processing circuitry 110 may determine a first flow measure using the first flow signal at 802. Processing circuitry 110 may evaluate if a flow threshold (e.g., F-T) has been determined at 804 and determine a flow threshold at 806 using the first flow measure (e.g., via “N” at 804). Hence, the flow threshold may be indicative of a blood flow (e.g., a cerebral blood flow) of patient 102 that might be expected when patient 102 is assessed to be (e.g., by a clinician) in a medically stable state. Processing circuitry 110 may substantially monitor patient 102 subsequent to establishing the flow threshold by determining a flow parameter and comparing the flow parameter to the flow threshold.

In examples, processing circuitry 110 determines the flow parameter by continuing to receive the flow signal subsequent to the determination of the flow threshold (e.g., F-T). For example, when F-T has been determined, such that processing circuitry 110 follows “Y” at 804, processing circuitry 110 may return to 801 to continue to receive the flow signal. Processing circuitry 110 may proceed through one or more of 801, 802, and/or 804 to determine the flow parameter. In some examples, the flow parameter is based on a flow signal caused by a second acoustic response of blood within or in the vicinity of anatomical volume 106 and caused by light emitter device 118. In examples, light emitter device 118 illuminates blood within anatomical volume 106 with light including the first wavelength and the second wavelength during the monitoring period to cause the second acoustic response.

Processing circuitry 110 may compare the flow parameter (e.g., F-P) and the flow threshold (e.g., F-T) to assess a condition of patient 102 during the monitoring period relative to the condition of patient 102 during the initial period (810). Processing circuitry 110 may generate a flow alert (e.g., one of the first flow alert or the second flow alert) based on the comparison of the flow parameter and the flow threshold. In examples, processing circuitry 110 issues the first flow alert (“B”) if the flow parameter is less than the flow threshold (e.g., if F-P is less than F-T). In some examples, processing circuitry 110 issues the second flow alert (“C”) if the flow parameter is greater than the flow threshold (e.g., if F-P is less than F-T). Processing circuitry 110 may continue to receive the flow signal and determine subsequent flow parameters (e.g., via 801, 802, 804, and/or 808), and compare the subsequent flow parameter to the flow threshold. Processing circuitry 110 may continue to determine subsequent flow parameters on a substantially ongoing basis to continue to monitor the condition of patient 102.

FIG. 8B depicts another example technique by which processing circuitry 110 may compare the flow parameter to the flow threshold. Processing circuitry 110 may use the technique of FIG. 8B instead of or in addition to the technique of FIG. 8A. Processing circuitry 110 may be configured to compare the flow parameter to the flow threshold using other techniques in other examples.

Processing circuitry 110 may determines the flow threshold by receiving the flow over the initial period when patient 102 is assessed (e.g., by a clinician) to be medically stable. In examples, the flow threshold is based on an electrical impedance spectrum determined using stent 104 (e.g., conductive fabric 174) during the initial period.

For example, processing circuitry 110 may determine the flow threshold using an initial flow signal received from stent 104 during the initial period at 812. The initial flow signal may be caused by an initial acoustic response caused by light emitter device 118 during the initial period (e.g., caused by illumination of the blood within anatomical volume 106 with light including the first wavelength and the second wavelength). In examples, the initial flow signal is indicative of an impedance of some portion of conductive fabric 174 (e.g., electrode 172, 174).

Processing circuitry 110 may determine an electrical impedance spectrum Z-0 (“Z-0”) using the initial signal at 812. In examples, Z-0 is an impedance spectrum including a range of from about 10 Hz to about 1 megahertz (MHz). Processing circuitry 110 may determine an initial flow measure using the initial flow signal at 814. In examples, processing circuitry 110 determines the initial flow measure by determining a power spectral density amplitude (“PSD”) of Z-0. Processing circuitry 110 may evaluate if a flow threshold (e.g., Z-T) has been determined at 816 and determine a flow threshold at 818 using the initial flow measure (e.g., via “N” at 816). Hence, the flow threshold Z-T may be indicative of a blood flow (e.g., a cerebral blood flow) of patient 102 that might be expected when patient 102 is assessed to be (e.g., by a clinician) in a medically stable state. Processing circuitry 110 may substantially monitor patient 102 subsequent to establishing the flow threshold Z-T by determining a flow parameter Z-P and comparing the flow parameter Z-P to the flow threshold Z-T.

In examples, processing circuitry 110 determines the flow parameter Z-T by continuing to receive the flow signal subsequent to the determination of the flow threshold Z-T. For example, when Z-T has been determined, such that processing circuitry 110 follows “Y” at 816, processing circuitry 110 may return to 812 to continue to receive the flow signal. Processing circuitry 110 may proceed through one or more of 812, 814, and/or 816 to determine the flow parameter Z-P. In some examples, the flow parameter Z-P is based on a flow signal caused by a by a subsequent acoustic response caused by light emitter device 118 during the monitoring period. In examples, the subsequent flow signal is indicative of an impedance of some portion of conductive fabric 174 (e.g., electrode 172, 174). Processing circuitry 110 may determine the flow parameter Z-P by determining a Z-0 and a PSD of the subsequent flow signal.

Processing circuitry 110 may compare the flow parameter (e.g., Z-P) and the flow threshold (e.g., Z-T) to assess a condition of patient 102 during the monitoring period relative to the condition of patient 102 during the initial period (822). Processing circuitry 110 may generate a flow alert (e.g., one of the first flow alert or the second flow alert) based on the comparison of Z-P and Z-T. In examples, processing circuitry 110 issues the first flow alert (“B”) if the flow parameter is less than the flow threshold (e.g., if Z-P is less than Z-T). In some examples, processing circuitry 110 issues the second flow alert (“C”) if the flow parameter is greater than the flow threshold (e.g., if Z-P is greater than Z-T). Processing circuitry 110 may continue to receive the flow signal and determine subsequent flow parameters (e.g., via 812, 814, 8816, and/or 820), and compare the subsequent flow parameter to the flow threshold. Processing circuitry 110 may continue to determine subsequent flow parameters on a substantially ongoing basis to continue to monitor the condition of patient 102.

Processing circuitry 110 may be configured to provide a stroke alert indicating that patient 102 may be experiencing or may have experienced a stroke based on the EEG alert, the first oxygen alert, the second oxygen alert, the third oxygen alert, the first flow alert, and/or the second flow alert. In some examples, processing circuitry 110 is configured to provide the stroke alert when the processing circuitry initiates all of the EEG alert, one or more of the first oxygen alert, the second oxygen alert, and/or the third oxygen alert, and one or more of the first flow alert and/or the second flow alert.

In examples, processing circuitry 110 is configured to indicate a type of stroke based on the EEG alert, the first oxygen alert, the second oxygen alert, the third oxygen alert, the first flow alert, and/or the second flow alert. For example, processing circuitry 110 may be configured to indicate patient 102 may have experienced a first type of stroke (e.g., an ischemic stroke) when processing circuitry 110 initiates a first combination of the EEG alert, the first oxygen alert, the second oxygen alert, the third oxygen alert, the first flow alert, and/or the second flow alert. Processing circuitry 110 may be configured to indicate patient 102 may have experienced a second type of stroke (e.g., a hemorrhagic stroke) when processing circuitry 110 initiates a second combination of the EEG alert, the first oxygen alert, the second oxygen alert, the third oxygen alert, the first flow alert, and/or the second flow alert different from the first combination.

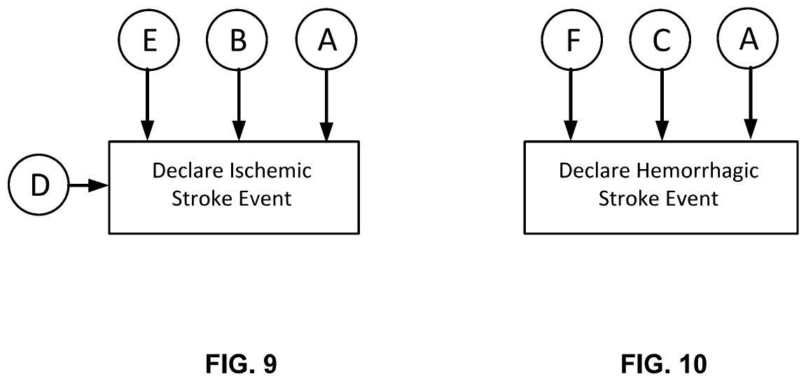

For example, FIG. 9 depicts a first example technique by which processing circuitry 110 may evaluate the EEG alert, the first oxygen alert, the second oxygen alert, the third oxygen alert, the first flow alert, and/or the second flow alert to declare a possible stroke event (e.g., an ischemic strike event). In some examples, processing circuitry 110 declares a possible stroke event (e.g., an ischemic stroke event) when processing circuitry initiates the EEG alert (e.g., “A” at FIG. 5), either or both of the second oxygen alert (e.g., “E” at FIG. 6) and the third oxygen alert (e.g., “D” at FIG. 7), and the first flow alert (e.g., “B” at FIG. 8A, 8B).

FIG. 10 depicts a second example technique by which processing circuitry 110 may evaluate the EEG alert, the first oxygen alert, the second oxygen alert, the third oxygen alert, the first flow alert, and/or the second flow alert to declare another possible stroke event (e.g., a hemorrhagic stroke event). In some examples, processing circuitry 110 declares another possible stroke event (e.g., a hemorrhagic stroke event) when processing circuitry initiates the EEG alert (e.g., “A” at FIG. 5), the first oxygen alert (e.g., “F” at FIG. 6), and the second flow alert (e.g., “C” at FIG. 8A,8B).

Processing circuitry 110 may be configured to declare a stroke event based on any type of EEG alert communicated by processing circuitry 110 in response to a comparison of any type of EEG parameter indicative of an EEG of patient 102 during a monitoring period with any type of EEG threshold indicative of an EEG of patient 102 during an initial period. In examples, the EEG parameter is determined and/or expressed using a particular measurement unit and/or methodology (e.g., a DAR) indicative of the EEG of patient 102 during the monitoring period and the EEG threshold is determined and/or expressed using the particular measurement unit and/or methodology indicative of the EEG of patient 102 during the monitoring period. In examples, the oxygen parameter is determined and/or expressed using as a specific type of measurement unit and/or methodology (e.g., an SjVO2) indicative of the oxygen level of patient 102 during the monitoring period and the oxygen threshold is determined and/or expressed using the specific type of measurement unit indicative of the oxygen level of patient 102 during the monitoring period. In examples, the flow parameter is determined and/or expressed using a given type of measurement unit and/or methodology (e.g., a mass flow rate) indicative of the flow rate of blood of patient 102 during the monitoring period and the flow threshold is determined and/or expressed using the given type of measurement unit indicative of the flow rate of patient 102 during the monitoring period. Further, the initial period may be comprised of a first initial period portion during which the EEG threshold is determined, a second initial period portion during which the oxygen threshold is determined, and/or a third initial period portion during which the flow threshold is determined, although this is not required. In some examples, the EEG threshold, the oxygen threshold, and/or the flow threshold may be determined substantially in parallel during the initial period. The monitoring period may be comprised of a first monitoring period portion during which the EEG parameter is determined, a second monitoring period portion during which the oxygen parameter is determined, and/or a third monitoring period portion during which the flow threshold is determined, although this is not required. In some examples, the EEG parameter, the oxygen parameter, and/or the flow parameter may be determined substantially in parallel during the monitoring period. The first monitoring period portion may be chronologically subsequent to the first initial period portion. The second monitoring period portion may be chronologically subsequent to the second initial period portion. The third monitoring period portion may be chronologically subsequent to the third initial period portion.

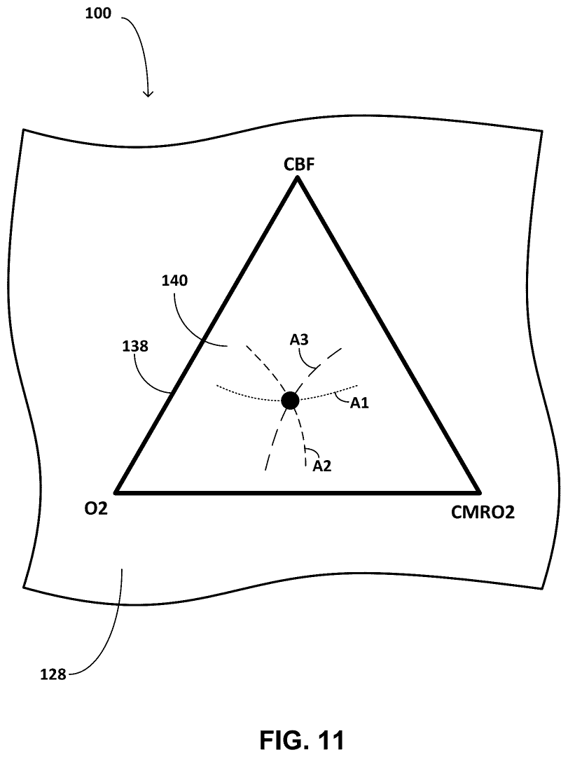

FIG. 11 is a schematic plan view of a portion of display device 128 of medical system 100. Display device 128 includes visual display 138 configured to provide a visual indication indicative of one or more or the EEG parameter, the oxygen parameter, the flow parameter, and/or the metabolic rate. Visual display 138 may be configured such that the visual indication of the one or more or the EEG parameter, the oxygen parameter, the flow parameter, and/or the metabolic rate provides an indication of the neurovascular coupling of patient 102.

In examples, display device 128 is configured to receive a flow input signal (e.g., via comm link 132 (FIG. 1)) indicative of the flow parameter from processing circuitry 110. Display device 128 may be configured to receive an oxygen input signal (e.g., via comm link 132 (FIG. 1)) indicative of the oxygen parameter from processing circuitry 110. Display device 128 may be configured to receive a metabolic rate input (e.g., via comm link 132 (FIG. 1)) indicative of the metabolic rate from processing circuitry 110. Visual display 138 may be configured to display a visual indication indicative of one or more of the flow input signal, the oxygen input signal, and/or the metabolic rate input signal.

For example, in some examples, visual display 138 is configured to provide the flow visual indication, the oxygen visual indication, and the metabolic rate visual indication on triangle graph 140. In examples, triangle graph 140 defines a first vertex (“CBF”) corresponding to the flow input signal, a second vertex (“O2”) corresponding to the oxygen input signal, and a third vertex (“CMRO2”) corresponding to the metabolic rate input signal. Visual display 138 may be configured to provide the flow visual indication, the oxygen visual indication, and the metabolic rate visual indication as a point on triangle graph 140. For example, visual display 138 and/or processing circuitry 110 may be configured to determine a first arc A1 defining a first distance from the first vertex, wherein the first distance is indicative of the flow visual indication. Visual display 138 and/or processing circuitry 110 may be configured to determine a second arc A2 defining a second distance from the second vertex, wherein the second distance is indicative of the oxygen visual indication. Visual display 138 and/or processing circuitry 110 may be configured to determine a third arc A3 defining a third distance from the third vertex, wherein the third distance is indicative of the metabolic rate visual indication. Visual display 138 may be configured to define the point indicative of the flow visual indication, the oxygen visual indication, and the metabolic rate visual indication on triangle graph 140 based on an intersection of the first arc A1, the second arc A2, and the third arc A3. Visual display 138 may be configured to provide the flow visual indication, the oxygen visual indication, and the metabolic rate visual indication in other ways in other examples.

Processing circuitry 110 may include fixed function circuitry and/or programmable operating circuitry. In examples, processing circuitry 110 includes circuitry configured to perform one or more functions of operating circuitry, such as therapy delivery circuitry, sensing circuitry, processing circuitry, switching circuitry, communication circuitry, and/or other circuitries. Processing circuitry 110, as well as other processors, operating circuitry, controllers, control circuitry, and the like, described herein, may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, processing circuitry 110 includes multiple components, such as any combination of one or more microprocessors, one or more DSPs, one or more ASICs, or one or more FPGAs, as well as other discrete or integrated logic circuitry, and/or analog circuitry.

Functions attributed to processing circuitry 110 may be embodied as software, firmware, hardware or any combination thereof. Processing circuitry 110 may include, for instance, a variety of capacitors, transformers, switches, and the like configured to perform the functions of processing circuitry 110. In examples, processing circuitry 110 may be configured to communicate with another device 134 (e.g., via a communication link 133), such as a patient input/output device, a clinician input/output device, a networked computing device, a computer network, and/or others. Processing circuitry 110 may include any suitable hardware, firmware, software or any combination thereof for communicating with device 134. In examples, processing circuitry 110 and/or other circuitry of medical system 100 is configured to deliver stimulation signals to and/or receive sensing signals from electrode 170, 172, conductive fabric 174, first material 180, and/or second material 184, and/or other electrodes and/or sensors within medical system 100 or external to medical system 100. Processing circuitry 110 may be configured to provide electrical signals to electrode 170, 172, conductive fabric 174, first material 180, and/or second material 184, and/or other electrodes within medical system 100. Processing circuitry 110 may be configured to receive electrical signals, e.g., signals indicative of the EEG signal, the oxygen signal, and/or the flow signal, from electrode 170, 172, conductive fabric 174, first material 180, and/or second material 184, and/or other electrodes within medical system 100.

Medical system 100 may include a power source configured to deliver operating power to various components of medical system 100, such as stent 104 processing circuitry 110, light source 122, and/or other systems and/or components of medical system 100. The power source may include a small rechargeable or non-rechargeable battery and a power generation circuit to produce the operating power. In some examples, recharging may be accomplished through proximal inductive interaction between an external charger and an inductive charging coil within medical system 100. In some examples, power requirements may be small enough to allow medical system 100 to utilize patient motion and implement a kinetic energy-scavenging device to trickle charge a rechargeable battery. In other examples, traditional batteries may be used for a limited period of time.

Medical system 100 (e.g., processing circuitry 110) can also include a memory configured to store program instructions, such as software, which may include one or more program modules, which are executable by processing circuitry 110. The program instructions may be embodied in software and/or firmware. The memory can include any volatile, non-volatile, magnetic, optical, or electrical media, such as a random access memory (RAM), read-only memory (ROM), non-volatile RAM (NVRAM), electrically-erasable programmable ROM (EEPROM), ferroelectric RAM (FRAM), flash memory, or any other digital media. In some examples, the memory includes computer-readable instructions that, when executed by processing circuitry 110 cause processing circuitry 110 to perform various functions described herein and/or other functions of processing circuitry 110.

Housing 126 may enclose processing circuitry 110 and/or other circuitry within medical system 100. Housing 126 may be configured to fluidly isolate processing circuitry 110 and/or other circuitry from an environment in contact with an exterior surface of housing 126. In examples, housing 126 is configured to hermetically seal an enclosure holding processing circuitry 110 and/or other circuitry. Housing 126 may be configured to define shapes that are easily accepted by the patient's body while minimizing patient discomfort. For example, portions of housing 126 may define a substantially cylindrical shape with cylindrical sidewalls. In other examples, portions of housing 126 may define substantially rectangular or other non-cylindrical shapes. Housing 126 may define shapes in which corners and edges are designed with relatively large radii, in order to present a housing having smoothly contoured exterior surfaces.

Comm link 114, 115, 116, 117, 119, 132, 133 may be hard-line and/or wireless communications links. In some examples, comm link 114, 115, 116, 117, 119, 132, 133 may comprise some portion of processing circuitry 110. In some examples, comm link 114, 115, 116, 117, 119, 132, 133 comprise a wired connection, a wireless Internet connection, a direct wireless connection such as wireless LAN, Bluetooth™, Wi-Fi™, and/or an infrared connection. Comm link 114, 115, 116, 117, 119, 132, 133 may utilize any wireless or remote communication protocol.

As used here, when a first portion of a system (e.g., medical system 100) supports a second portion of the system, this means that when the second portion causes a first force to be exerted on the first portion, the first portion causes a second force to be exerted on the second portion in response to the first force. The first force and/or second force may be a contact force and/or an action-at-a-distance force. For example, first force and/or second force may be mechanical force, a magnetic force, a gravitational force, or some other type of force. The first portion of the system may be a portion of the system or a portion of a component of the system. The second portion of the system may be another portion of the system or another portion of the same component or a different component. In some examples, when the first portion of the system supports the second portion of the system, this may mean the second portion is mechanically supported by and/or mechanically connected to the first portion.

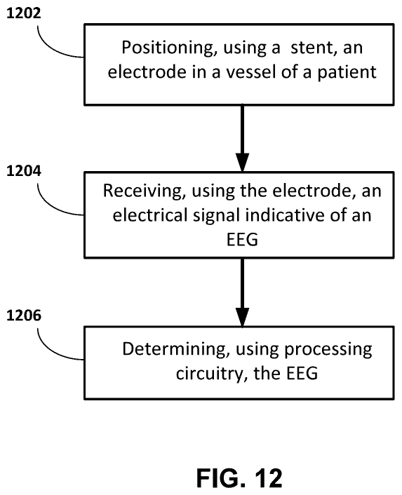

A technique for monitoring a patient 102 using a stent 104 is illustrated in FIG. 12. Although the technique is described mainly with reference to medical system 100 of FIGS. 1-11, the technique may be applied to other medical systems in other examples.

The technique includes supporting, using a skeleton 112 of stent 104 positioned within a vessel 142 of patient 102, electrode 170, 172 (1202). Skeleton 112 may define a stent lumen 105 when skeleton 112 is positioned within vessel 142. In examples, supporting electrode 170, 172 includes supporting a conductive elongate fiber 182, 188 comprising one or more of electrode 170, 172. Stent 104 may allow blood in vessel 142 to flow in a direction along a longitudinal axis L extending from a stent proximal portion 148 to a stent distal portion 146 when skeleton 112 is positioned in vessel 142.

The technique may include transitioning, using delivery system 107, skeleton 112 from a deployment configuration defining a first cross-sectional dimension D1 to an expanded configuration defining a second cross-sectional dimension D2 greater than first cross-sectional dimension D1 when skeleton 112 is positioned in vessel 142. In some examples, the technique includes transitioning, using delivery system 107, skeleton 112 from the expanded configuration to the deployment configuration when skeleton 112 is positioned in vessel 142. In some examples, stent 104 contacts a vessel wall 144 of vessel 142 when skeleton 112 establishes the expanded configuration. In some examples, stent 104 defines stent lumen 105 using a conductive fabric 174 supported by skeleton 112 and surrounding longitudinal axis L.

The technique includes receiving, using electrode 170, 172 an electrical signal indicative of an EEG of the patient 102 when skeleton 112 positions in vessel 142 (1204). The technique includes providing, using stent 104, a EEG signal indicative of the electrical signal to processing circuitry 110 configured to determine an EEG of patient 102 using the EEG signal (1206). In examples, providing the EEG signal to processing circuitry 110 comprises providing the signal as a unipolar electrical signal. Processing circuitry 110 may determine an EEG of patient 102 using the EEG signal.

In examples, stent 104 provides an oxygen signal indicative of an oxygen level of the blood within stent lumen 105 and/or vessel 142 to processing circuitry 110. Stent 104 may provide a flow signal indicative of a flow rate of the blood through stent lumen 105 and/or vessel 142 to processing circuitry 110. Stent 104 may provide the oxygen signal using one of a first material 180 or a second material 184 and provide the flow signal using the other of first material 180 or second material 184. Stent 104 may produce the oxygen signal and/or the flow signal using one or more acoustic waves caused by a photoacoustic response of blood within stent lumen 105 and/or vessel 142. In examples, a light emitter device 118 supported by skeleton 112 emits light to provoke the photoacoustic response. Stent 104 may produce, using first material 180, a first signal caused by impingement of the one or more acoustic waves with the first material. Stent 104 may produce, using second material 184, a second signal caused by impingement of the one or more acoustic waves with the second material.

Stent 104 may communicate the flow signal and the oxygen signal to processing circuitry 110. Processing circuitry 110 may determine an oxygen level of blood within stent lumen 105 and/or vessel 142 using the oxygen signal. Processing circuitry 110 may determine an flow rate of blood within stent lumen 105 and/or vessel 142 using the flow signal. In some examples, processing circuitry 110 determine the flow rate using an alteration of an impedance of at least one of conductive fabric 174 or another additional material supported by stent 104.

Processing circuitry 110 may determine an EEG parameter using at least one of the EEG or the EEG signal. Processing circuitry 110 may determine an oxygen parameter using at least one of the oxygen level or the one of the first signal or the second signal used to determine the oxygen level. Processing circuitry 110 may determine a flow parameter using at least one of the flow rate or the flow signal. Processing circuitry 110 may compare the EEG parameter to an EEG threshold, compare the oxygen parameter to an oxygen threshold, and/or compare the flow parameter to a flow threshold. In examples, processing circuitry 110 provides a stroke indication when at least one of the EEG parameter satisfies the criteria based on the EEG threshold, the oxygen parameter satisfies the criteria based on the oxygen parameter, or the flow parameter satisfies the criteria based on the flow threshold.

In some examples, processing circuitry 110 determines the EEG parameter and/or the EEG threshold by determining at least an alpha wave of the EEG and a delta wave of the EEG. Processing circuitry 110 may generate an EEG alert based on the comparison of the EEG parameter and the EEG threshold. In examples, processing circuitry 110 determines the EEG threshold during an initial period and determines the EEG parameter during a monitoring period subsequent to the initial period.

Processing circuitry 110 may determine the oxygen threshold by determine a change in the oxygen signal during the initial period. Processing circuitry 110 may determine the oxygen parameter by determining a change in the oxygen signal during the monitoring period. Processing circuitry 110 may generate an oxygen alert based on the comparison of the oxygen parameter and the oxygen threshold. In examples, the oxygen alert is one of a first oxygen alert, a second oxygen alert, or a third oxygen alert. Processing circuitry 110 may determine the flow threshold using flow signals received during the monitoring period and determine the flow parameter using flow signal received during the monitoring period. Processing circuitry 110 may generate an flow alert based on the comparison of the flow parameter and the flow threshold. In examples, the flow alert is one of a first flow alert or a second flow alert. In examples, the processing circuitry determines a metabolic rate using a first oxygen parameter component, a second oxygen parameter component, and the flow parameter.

Processing circuitry 110 may issue a stroke alert in response to generating at least one of the EEG alert, the oxygen alert, or the flow alert. In examples, processing circuitry 110 provides, based on combinations of the EEG alert, the oxygen alert, and the flow alert, one of an ischemic stroke alert or a hemorrhagic stroke alert. In some examples, processing circuitry 110 issues the ischemic stroke alert in response to generating the EEG alert, at least one of the first oxygen alert or the third oxygen alert, and the first flow alert. In examples, processing circuitry 110 issues the hemorrhagic stroke alert in response to generating the EEG alert, the second oxygen alert, and the second flow alert.

In some examples, processing circuitry 110 provides a flow input signal indicative of the flow parameter to a display device 128. Processing circuitry 110 may provide an oxygen input signal indicative of the oxygen parameter to display device 128. Processing circuitry 110 may provide a metabolic rate input indicative of the metabolic rate to display device 128. Display device 128 may provide a flow visual indication indicative of the flow input signal on a visual display 138 of display device 128, provide oxygen visual indication indicative of the oxygen input signal on visual display 138, and/or provide a metabolic rate visual indication indicative of the metabolic rate input signal on visual display 138. In examples, display device 128 is configured to provide the flow visual indication, the oxygen visual indication, and the metabolic rate visual indication on a triangle graph 140 having a first vertex corresponding to the flow input signal, a second vertex corresponding to the oxygen input signal, and a third vertex corresponding to the metabolic rate input signal.

Various examples of the disclosure have been described. Any combination of the described systems, operations, or functions is contemplated. These and other examples are within the scope of the following claims.

What is claimed is:

1. A medical system including a stent configured to expand within a blood vessel a patient, the stent comprising:
a skeleton configured to define a lumen when the skeleton is positioned within the blood vessel;
a conductive material supported by the skeleton, wherein the conductive material is configured to receive and conduct an electrical signal indicative of a electroencephalogram (EEG) of the patient when the skeleton positions within the blood vessel;
a light emitter device supported by the skeleton and positioned within the lumen, wherein the light emitter device is configured to cause a photoacoustic response of some portion of the blood in the vessel when the light emitter device projects light into the portion of the blood, and wherein the photoacoustic response is indicative of an oxygen level of the portion of the blood; and
a first material supported by the skeleton, wherein the first material is configured to develop a first electric potential in response to a first mechanical stress generated by impingement of a pressure wave on the first material caused by the photoacoustic response,
wherein the stent is configured to communicate a EEG signal indicative of the electrical signal from the conductive material to processing circuitry, communicate a first signal indicative of the first electric potential from the first material to the processing circuitry, and communicate a flow signal indicative of a blood flow rate in the blood vessel to the processing circuitry, and
wherein the stent is configured to allow the blood in the vessel to flow in a direction along a longitudinal axis extending from a proximal portion of the stent to a distal portion of the stent when the skeleton positions within the blood vessel.

2. The medical system of claim 1, further comprising a second material supported by the stent, wherein the second material is configured to develop a second electric potential in response to a second mechanical stress generated by impingement of at least one pressure wave on the second material caused by the photoacoustic response, and wherein the flow signal is indicative of the second electrical charge from the second material to the processing circuitry.

3. The medical system of claim 1, wherein at least one of the conductive material, the first material, or an additional material supported by the stent is configured to alter its impedance in response to impingement of the pressure wave caused by the photoacoustic response, and wherein the flow signal is indicative of the alteration of the impedance.

4. The medical system of claim 1, wherein the portion proximal is configured to define a proximal opening to the lumen and the distal portion is configured to define a distal opening to the lumen, and wherein the stent is configured to allow the blood to flow from the proximal opening to the distal opening when the skeleton is positioned within the blood vessel.

5. The medical system of claim 4, wherein the skeleton is configured to radially expand in a direction substantially perpendicular to the longitudinal axis when the skeleton transitions from a deployment configuration to an expanded configuration.

6. The medical system of claim 1, wherein the conductive material defines at least some portion of a lumen boundary of the lumen.

7. The medical system of claim 1, wherein the conductive material is configured to provide the EEG signal to the processing circuitry as a unipolar electrical signal.

8. The medical system of claim 1, further comprising the processing circuitry, wherein the processing circuitry is configured to:
determine the EEG using the EEG signal,
determine the oxygen level using an oxygen signal, wherein the oxygen signal is one of the first signal or the second signal, and
determine a flow rate indicative of a blood flow rate in the blood vessel using the flow signal.

9. The medical system of claim 8, wherein the flow signal is the other of the first signal or the second signal.

10. The medical system of claim 8, wherein the processing circuitry is configured to:
determine at least one of:
an EEG parameter using at least one of the EEG or the EEG signal,
an oxygen parameter using at least one of the oxygen level or the one of the first signal or the second signal used to determine the oxygen level, or
a flow parameter using at least one of the flow rate or the flow signal;
compare at least one of:
the EEG parameter to an EEG threshold,
the oxygen parameter to an oxygen threshold, or
the flow parameter to a flow threshold; and
provide a stroke indication when at least one of:
the EEG parameter satisfies a criteria based on the EEG threshold,
the oxygen parameter satisfies a criteria based on the oxygen parameter, or
the flow parameter satisfies a criteria based on the flow threshold.

11. The medical system of claim 10, wherein the processing circuitry is configured to provide the stroke indication when the EEG parameter satisfies the criteria based on the EEG threshold, the oxygen parameter satisfies the criteria based on the oxygen threshold, and the flow parameter satisfies the criteria based on the flow threshold.

12. The medical system of claim 10, wherein the processing circuitry is configured to issue a stroke alert in response to generating at least one of an EEG alert, a first oxygen alert, a second oxygen alert, a third oxygen alert, a first flow alert, or a second flow alert.

13. The medical system of claim 12, wherein the processing circuitry is configured to:
detect at least an alpha wave of the EEG and a delta wave of the EEG, wherein the EEG parameter comprises a first DAR-1, wherein the first DAR-1 is indicative of a ratio of a delta wave power of a first power spectral density of the EEG signal within a first time period to a first alpha wave power of the first power spectral density, and wherein the EEG threshold comprises a primary DAR-T1, wherein the primary DAR-T1 is indicative of a ratio of a delta wave power of a primary power spectral density within a primary time period to an alpha wave power of the primary power spectral density, and wherein the primary time period chronologically precedes the first time period; and
generate the EEG alert based on a parameter determined using at least one of the first DAR-1 or the primary DAR-T1.

14. The medical system of claim 12, wherein the processing circuitry is configured to generate at least one of:
the first oxygen alert if a time rate of change of a difference ΔO2-P is greater than a time rate of change of a difference ΔO2-T, wherein the difference ΔO2-T indicative of a difference between a first O2 threshold level and a second O2 threshold level, wherein the oxygen threshold is based on the difference ΔO2-T, and wherein the difference ΔO2-P indicative of a difference between a first O2 parameter level and a second O2 parameter level, and wherein the oxygen parameter is based on the difference ΔO2-P;
the second oxygen alert if the time rate of change of the difference ΔO2-P is less than the time rate of change of the difference ΔO2-T; or
the third oxygen alert if a sensed BMR ratio is less than a threshold BMR ratio, wherein the threshold BMR ratio is indicative of a ratio of a power spectral density of a first B wave to a power spectral density of a first M wave, and wherein the sensed BMR ratio is indicative of a ratio of a power spectral density of a second B wave to a power spectral density of a second M wave.

15. The medical system of 12, wherein the processing circuitry is configured to:
determine the flow threshold using one or more first flow signals, wherein each of the one or more first flow signals are indicative of the at least one of the flow rate or the other of the first signal or the second signal used to determine the flow rate;
determine the flow parameter using one or more second flow signals, wherein each of the one or more second flow signals are indicative of the at least one of the flow rate or the other of the first signal or the second signal used to determine the flow rate, and
generate, if the flow parameter is greater than the flow threshold, the first flow alert; and
generate, if the flow parameter is less than or equal to the flow threshold, the second flow alert.

16. A medical system including a stent configured to expand within a blood vessel of a patient, the stent comprising:
a skeleton configured to define a lumen when the skeleton is positioned within the blood vessel, wherein the stent is configured to allow the blood in the vessel to flow in a direction along a longitudinal axis extending from a proximal portion of the stent to a distal portion of the stent when the skeleton positions within the blood vessel;
one or more conductive elongate fibers supported by the skeleton and extending from the proximal portion to the distal portion, wherein the one or more conductive elongate fibers are configured to receive an electrical signal indicative of a electroencephalogram of the patient when the skeleton positions within the blood vessel; and
processing circuitry configured to receive a signal indicative of the electrical signal from the one or more conductive elongate fibers, wherein the processing circuitry is configured to determine the electroencephalogram using the signal.

17. The medical system of claim 16
wherein the skeleton is configured to establish a deployment configuration defining a first cross-sectional dimension and establish an expanded configuration defining a second cross-sectional dimension greater than the first cross-sectional dimension,
wherein the skeleton is configured to transition from the deployment configuration to the expanded configuration when the skeleton is positioned within the blood vessel,
wherein the portion proximal is configured to define a proximal opening to the lumen and the distal portion is configured to define a distal opening to the lumen at least when the skeleton establishes the expanded configuration, and
wherein the stent is configured to allow the blood to flow from the proximal opening to the distal opening when the skeleton is positioned within the blood vessel.

18. The medical system of claim 16, wherein the one or more conductive elongate fibers comprise a conductive fabric supported by the skeleton and substantially surrounding the longitudinal axis

19. A method, comprising
supporting, using a skeleton of a stent configured to define a lumen when the skeleton is positioned within a blood vessel of a patient, one or more conductive elongate fibers extending from a proximal portion of the stent to a distal portion of the stent, wherein the stent is configured to allow the blood in the blood vessel to flow in a direction along a longitudinal axis extending from the proximal portion to the distal portion when the skeleton is positioned in the blood vessel;
receiving, using the one or more conductive fibers, an electrical signal indicative of a electroencephalogram (EEG) of the patient when the skeleton positions in the blood vessel; and
providing, using the stent, a EEG signal indicative of the electrical signal to processing circuitry configured to determine the electroencephalogram using the EEG signal.

20. The method of claim 19, further comprising:
transitioning the skeleton from a deployment configuration defining a first cross-sectional dimension to an expanded configuration defining a second cross-sectional dimension greater than the first cross-sectional dimension when the skeleton is positioned in the blood vessel; and
contacting, using the stent, a vessel wall of the blood vessel when the skeleton establishes the expanded configuration.
